(19) United States
(12) Patent Application Publication
(10) Pub. No.: US 2023/0133077 A1
(43) Pub. Date: May 4, 2023

(54) RADIO-FREQUENCY COIL ASSEMBLIES OF A MAGNETIC RESONANCE SYSTEM AND ASSEMBLING METHODS THEREOF

(71) Applicant: GE PRECISION HEALTHCARE LLC, Wauwatosa, WI (US)

(72) Inventors: Mark Giancola, Chesterland, OH (US); Clyve Konrad Rosales Follante, Twinsburg, OH (US); Tony T. Thomas, Twinsburg, OH (US); Gustavo Andres Chacon Rojas, Cleveland Heights, OH (US); Kolman Juhasz, Rocky River, OH (US); Fraser J.L. Robb, Aurora, OH (US); Yun-Jeong Stickle, Solon, OH (US); Robert Steven Stormont, Hartland, WI (US); Chris Puehn, Mayfield Heights, OH (US); Jana M. Vincent, Aurora, OH (US)

(21) Appl. No.: 17/514,879

(22) Filed: Oct. 29, 2021

(51) Int. Cl. A61B 5/055; G01R 33/34; G01R 33/36; A61B 5/00

(52) U.S. Cl. CPC A61B 5/055; G01R 33/34007; G01R 33/36; A61B 5/004; G01R 33/341

(57) ABSTRACT

A radio frequency (RF) coil assembly for a magnetic resonance (MR) system is provided. The RF coil assembly includes an RF coil array and a substrate assembly. The RF coil array includes one or more RF coils each RF coil including a coil loop that includes a wire conductor, the wire conductor formed into the coil loop. The substrate assembly includes a first substrate layer and a second substrate layer, wherein the first substrate layer is coupled with the second substrate layer at seams without a separately-provided fastening mechanism, wherein the RF coil array is positioned between the first substrate layer and the second substrate layer.

### BACKGROUND

The field of the disclosure relates generally to a magnetic resonance (MR) system, and more particularly, to radio frequency (RF) coil assemblies for an MR system.

Magnetic resonance imaging (MRI) has proven useful in diagnosis of many diseases. MRI provides detailed images of soft tissues, abnormal tissues such as tumors, and other structures, which cannot be readily imaged by other imaging modalities, such as computed tomography (CT). Further, MRI operates without exposing patients to ionizing radiation experienced in modalities such as CT and x-rays.

In MRI, a radio-frequency (RF) coil assembly is used to detect MR signals emitted from a subject. As such, the RF coil assembly is desirable to be light-weighted and flexible to conform to the anatomy of the subject for comfort and image quality. Known RF coil assemblies is disadvantaged in some aspects and improvements are desired.

### BRIEF DESCRIPTION

In one aspect, a radio frequency (RF) coil assembly for a magnetic resonance (MR) system is provided. The RF coil assembly includes an RF coil array and a substrate assembly. The RF coil array includes one or more RF coils each RF coil including a coil loop that includes a wire conductor, the wire conductor formed into the coil loop. The substrate assembly includes a first substrate layer and a second substrate layer, wherein the first substrate layer is coupled with the second substrate layer at seams without a separately-provided fastening mechanism, wherein the RF coil array is positioned between the first substrate layer and the second substrate layer.

In another aspect, a method of assembling an RF coil assembly of a medical imaging system is provided. The method includes positioning one or more coil loops on a first substrate layer, wherein each coil loop includes a wire conductor, the wire conductor formed into the coil loop. The method also includes positioning a second substrate layer over the one or more coil loops. The method further includes forming a substrate assembly that includes the first substrate layer and the second substrate layer by coupling the first substrate layer with the second substrate layer at seams without a separately-provided fastening mechanism.

In one more aspect, an RF coil assembly for a medical imaging system is provided. The RF coil assembly includes an RF coil array and a substrate array. The RF coil array includes one or more RF coils each RF coil including a coil loop that includes a wire conductor, the wire conductor formed into the coil loop. The substrate assembly includes a first substrate layer and a second substrate layer, wherein the first substrate layer is coupled with the second substrate layer at seams without a separately-provided fastening mechanism, wherein the RF coil array is positioned between the first substrate layer and the second substrate layer.

### DRAWINGS

Non-limiting and non-exhaustive embodiments are described with reference to the following figures, wherein like reference numerals refer to like parts throughout the various drawings unless otherwise specified.

FIG. 1 is a block diagram of a magnetic resonance (MR) system.

FIG. 2 is a block diagram of an exemplary radio frequency (RF) coil.

FIG. 3A is a schematic diagram of an exemplary RF coil shown in FIG. 2.

FIG. 3B is a schematic diagram of another exemplary RF coil shown in FIG. 2.

FIG. 3C is a cross-sectional view of an exemplary distributed capacitance coil loop of the RF coils shown in FIGS. 3A and 3B.

FIG. 4A is a schematic diagram of one more exemplary RF coil shown in FIG. 2.

FIG. 4B is a schematic diagram of one more exemplary RF coil shown in FIG. 2.

FIG. 4C is a cross-sectional view of an exemplary wire conductor used in the coil loop of the RF coils shown in FIGS. 4A and 4B

FIG. 5A is a top perspective view of an exemplary RF coil assembly.

FIG. 5B is a bottom perspective view of the RF coil assembly shown in FIG. 5A.

FIG. 5C is an exploded view of the RF coil assembly shown in FIG. 5A.

FIG. 5D is a block diagram of the RF coil assembly shown in FIG. 5A.

FIG. 6 is a cross-sectional view of a known RF coil assembly.

FIG. 7A is a top perspective view of the interior of the RF coil assembly shown in FIG. 5A.

FIG. 7B is a top view of the RF coil assembly shown in FIG. 7A.

FIG. 7C is a bottom view of the RF coil assembly shown in FIG. 7A.

FIG. 8A shows the RF coil assembly shown in FIG. 7A with the top substrate layer removed.

FIG. 8B shows the bottom substrate layer of the RF coil assembly shown in FIG. 8A.

FIGS. 9A-9C are schematic diagrams illustrating an exemplary method of assembling RF coil assemblies shown in FIGS. 2-5D and 7A-8B.

FIG. 9A shows that a coil array is positioned over a substrate layer.

FIG. 9B shows that another substrate layer is to be placed over the assembly shown in FIG. 9A.

FIG. 9C shows an RF coil assembly after welding of the components shown in FIG. 9B.

FIG. 10A is a top view of another exemplary RF coil assembly.

FIG. 10B is a perspective view of the RF coil assembly shown in FIG. 10A.

FIG. 10C is a side view of the RF coil assembly shown in FIG. 10A.

FIG. 11A is a top view of one more exemplary embodiment of RF coil assembly.

FIG. 11B is a schematic diagram of the RF coil assembly shown in FIG. 11A with a top substrate layer removed.

FIG. 11C is a perspective view of the RF coil assembly shown in FIG. 11A when being placed on a subject.

FIG. 12A is a top view of one more exemplary embodiment of an RF coil assembly.

FIG. 12B is a perspective view of the RF coil assembly shown in FIG. 12A with a flap in an opened position.

FIG. 12C is a perspective view of the RF coil assembly shown in FIG. 12A when being placed on a subject.

FIG. 12D is a perspective view of the RF coil assembly shown in FIG. 12C with a flap in an opened position.

FIG. 12E is a side view of the RF coil assembly shown in FIG. 12D.

FIG. 13A is a top view of one more exemplary embodiment of an RF coil assembly.

FIG. 13B is a perspective view of the RF coil assembly shown in FIG. 13A with a flap in an opened position.

FIG. 13C is a perspective view of the RF coil assembly shown in FIG. 13A when being placed on a subject.

FIG. 13D is a perspective view of the RF coil assembly shown in FIG. 13C with a flap in an opened position.

FIG. 13E is a schematic diagram of the RF coil assembly shown in FIG. 13D.

FIG. 14A is a perspective view of one more exemplary embodiment of an RF coil assembly.

FIG. 14B is a perspective view of the RF coil assembly shown in FIG. 14A with a flap in an opened position.

FIG. 14C is another perspective view of the RF coil assembly shown in FIG. 14B.

FIG. 15A is a perspective view of one more exemplary embodiment of an RF coil assembly.

FIG. 15B is a perspective view of the RF coil assembly shown in FIG. 15A with a flap in an opened position.

### DETAILED DESCRIPTION

The disclosure includes radio frequency (RF) coil assemblies for use in magnetic resonance (MR) systems in scanning a subject. As used herein, a subject is a human, an animal, a phantom, or any object scanned by a medical imaging system. MR imaging is described as an example only. The assemblies, systems, and methods described herein may be used for MR spectroscopy. MR systems are described as an example only. The RF coil assemblies and method of assembling RF coil assemblies described herein may be used for medical imaging systems other than MR systems, such as positron emission tomography (PET)-MR systems. Method aspects of assembling and using the RF coil assemblies will be in part apparent and in part explicitly discussed in the following description.

In MR imaging (MRI), a subject is placed in a magnet. When the subject is in the magnetic field generated by the magnet, magnetic moments of nuclei, such as protons, attempt to align with the magnetic field but precess about the magnetic field in a random order at the nuclei's Larmor frequency. The magnetic field of the magnet is referred to as B0 and extends in the longitudinal or z direction. In acquiring an MRI image, a magnetic field (referred to as an excitation field B1), which is in the x-y plane and near the Larmor frequency, is generated by a RF coil and may be used to rotate, or “tip,” the net magnetic moment Mz of the nuclei from the z direction to the transverse or x-y plane. A signal, which is referred to as an MR signal, is emitted by the nuclei, after the excitation signal B1 is terminated. To use the MR signals to generate an image of a subject, magnetic field gradient pulses (Gx, Gy, and Gz) are used. The gradient pulses are used to scan through the k-space, the space of spatial frequencies or inverse of distances. A Fourier relationship exists between the acquired MR signals and an image of the subject, and therefore the image of the subject can be derived by reconstructing the MR signals.

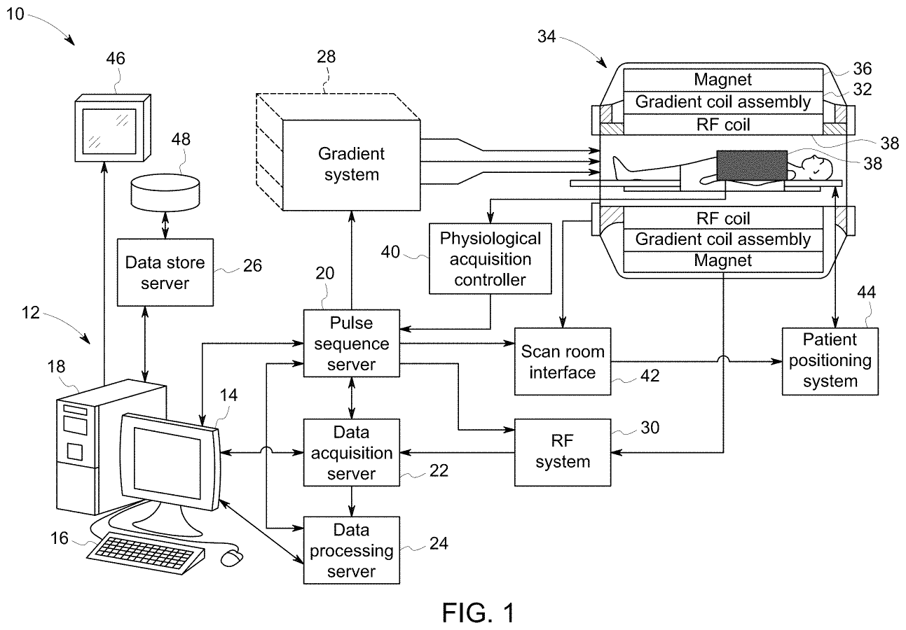

FIG. 1 illustrates a schematic diagram of an exemplary MR system 10. In the exemplary embodiment, MR system 10 includes a workstation 12 having a display 14 and a keyboard 16. Workstation 12 includes a processor 18, such as a commercially available programmable machine running a commercially available operating system. workstation 12 provides an operator interface that allows scan prescriptions to be entered into MR system 10. Workstation 12 is coupled to a pulse sequence server 20, a data acquisition server 22, a data processing server 24, and a data store server 26. Workstation 12 and each server 20, 22, 24, and 26 communicate with each other.

In the exemplary embodiment, pulse sequence server 20 responds to instructions downloaded from workstation 12 to operate a gradient system 28 and an RF system 30. The instructions are used to produce gradient and RF waveforms in MR pulse sequences. An RF coil assembly 38 and a gradient coil assembly 32 are used to perform the prescribed MR pulse sequence. RF coil assembly 38 may be a whole body RF coil. RF coil assembly 38 may also be a local RF coil assembly 38 that may be placed in proximity to the anatomy to be imaged, or a coil array that includes a plurality of coils.

In the exemplary embodiment, gradient waveforms used to perform the prescribed scan are produced and applied to gradient system 28, which excites gradient coils in gradient coil assembly 32 to produce the magnetic field gradients Gₓ, G_{y}, and G_{z} used for position-encoding MR signals. Gradient coil assembly 32 forms part of a magnet assembly 34 that also includes a polarizing magnet 36 and RF coil assembly 38.

In the exemplary embodiment, RF system 30 includes an RF transmitter for producing RF pulses used in MR pulse sequences. The RF transmitter is responsive to the scan prescription and direction from pulse sequence server 20 to produce RF pulses of a desired frequency, phase, and pulse amplitude waveform. The generated RF pulses may be applied to RF coil assembly 38 by RF system 30. Responsive MR signals detected by RF coil assembly 38 are received by RF system 30, amplified, demodulated, filtered, and digitized under direction of commands produced by pulse sequence server 20. RF coil assembly 38 is described as a transmit and receive coil such that RF coil assembly 38 transmits RF pulses and detects MR signals. In one embodiment, MR system 10 may include a transmit RF coil that transmits RF pulses and a separate receive coil that detects MR signals. A transmission channel of RF system 30 may be connected to a RF transmit coil and a receiver channel may be connected to a separate RF receive coil. Often, the transmission channel is connected to the whole body RF coil assembly 38 and each receiver section is connected to a separate local RF coil.

In the exemplary embodiment, RF system 30 also includes one or more RF receiver channels. Each RF receiver channel includes an RF amplifier that amplifies the MR signal received by RF coil assembly 38 to which the channel is connected, and a detector that detects and digitizes the I and Q quadrature components of the received MR signal. The magnitude of the received MR signal may then be determined as the square root of the sum of the squares of the I and Q components as in Eq. (1) below:

M=\sqrt{I²+Q²} (1);

and the phase of the received MR signal may also be determined as in Eq. (2) below:

$$φ = {tan}^{- 1} ( \frac{Q}{I} ) . \qquad ( 2 )$$

In the exemplary embodiment, the digitized MR signal samples produced by RF system 30 are received by data acquisition server 22. Data acquisition server 22 may operate in response to instructions downloaded from workstation 12 to receive real-time MR data and provide buffer storage such that no data is lost by data overrun. In some scans, data acquisition server 22 does little more than pass the acquired MR data to data processing server 24. In scans that need information derived from acquired MR data to control further performance of the scan, however, data acquisition server 22 is programmed to produce the needed information and convey it to pulse sequence server 20. For example, during prescans, MR data is acquired and used to calibrate the pulse sequence performed by pulse sequence server 20. Also, navigator signals may be acquired during a scan and used to adjust the operating parameters of RF system 30 or gradient system 28, or to control the view order in which k-space is sampled.

In the exemplary embodiment, data processing server 24 receives MR data from data acquisition server 22 and processes it in accordance with instructions downloaded from workstation 12. Such processing may include, for example, Fourier transformation of raw k-space MR data to produce two or three-dimensional images, the application of filters to a reconstructed image, the performance of a backprojection image reconstruction of acquired MR data, the generation of functional MR images, and the calculation of motion or flow images.

In the exemplary embodiment, images reconstructed by data processing server 24 are conveyed back to, and stored at, workstation 12. In some embodiments, real-time images are stored in a database memory cache (not shown in FIG. 1), from which they may be output to operator display 14 or a display 46 that is located near magnet assembly 34 for use by attending physicians. Batch mode images or selected real time images may be stored in a host database on disc storage 48 or on a cloud. When such images have been reconstructed and transferred to storage, data processing server 24 notifies data store server 26. workstation 12 may be used by an operator to archive the images, produce films, or send the images via a network to other facilities.

During a scan, RF coil array interfacing cables (not shown) may be used to transmit signals between RF coil assembly 38 and other aspects of MR system 10 (e.g., data acquisition server 22 and pulse sequence server 20), for example to control the RF coils and/or to receive signals from the RF coils. As described above, the RF coil assembly 38 may be a transmit coil that transmits RF excitation signals, or a receive coil that receives the MR signals emitted by the subject. In an example, the transmit and receive coils are a single mechanical and electrical structure or array of structures, with transmit/receive mode switchable by auxiliary circuitry. In other examples, a transmit coil and a receive coil may be independent structures that are physically coupled to each other via the RF system 30. For enhanced image quality, however, a receive coil is desirable to be mechanically and electrically isolated from the transmit coil. In such cases, the receive coil in the receive mode is electromagnetically coupled to and resonant with an RF “echo” that is stimulated by the transmit coil. On the other hand, during a transmit mode, the receive coil is electromagnetically decoupled from and therefore not resonant with the transmit coil, during transmission of the RF signal. Such decoupling averts a potential problem of noise produced within the auxiliary circuitry when the receive coil couples to the full power of the RF signal. Additional details regarding the uncoupling of the receive RF coil will be described below.

A traditional receive coil for MR includes several conductive intervals joined between themselves by capacitors. By adjusting the capacitors' values, the impedance of the RF coil may be brought to its minimal value, usually characterized by a low resistance. At a resonant frequency, stored magnetic and electric energy alternate periodically. Each conductive interval, due to its length and width, possesses a certain self-capacitance, where electric energy is periodically stored as static electricity. The distribution of this electricity takes place over the entire conductive interval length in the order of 5-15 cm, causing similar range electric dipole field. In proximity of a large dielectric load, self-capacitance of the intervals change, resulting in detuning of the coil. In case of a lossy dielectric, dipole electric field causes Joule dissipation characterized by an increase overall resistance observed by the coil.

Traditional RF coils may include acid etched copper traces or loops on printed circuit boards (PCBs) with lumped electronic components (e.g., capacitors, inductors, baluns, and resisters), matching circuitry, decoupling circuitry, and pre-amplifiers. Such a configuration is typically bulky, heavy, and rigid, and requires relatively strict placement of the coils relative to each other in an array to prevent coupling interactions among coil elements that may degrade image quality. As such, traditional RF coils and RF coil arrays lack flexibility and hence may not conform to subject anatomy, degrading imaging quality and subject comfort.

The RF coil used in the RF coil assemblies described herein includes a coil loop formed by wire conductors. In the case of two RF coils overlapping, the coupling electronics portion that couples with the coil loop of the RF coil has high blocking or source impedance, thereby minimizing mutual inductance coupling. Thin cross-sections of the wire conductor in the RF coil reduces the parasitic capacitance at the cross-overs or overlaps, and reduces other coupling such as electric field coupling and eddy current, in comparison to two traditional trace-based loops. The combination of high blocking impedance and thin cross-sections of the RF coil loop allows flexible placement of multiple coils into one RF coil assemblies over a finite area, while coupling between the RF coils is minimized and critical overlap between two loops is not required. Wire conductors also add flexibility to the coils, allowing the coil assembly to conform with a curved anatomy of a subject.

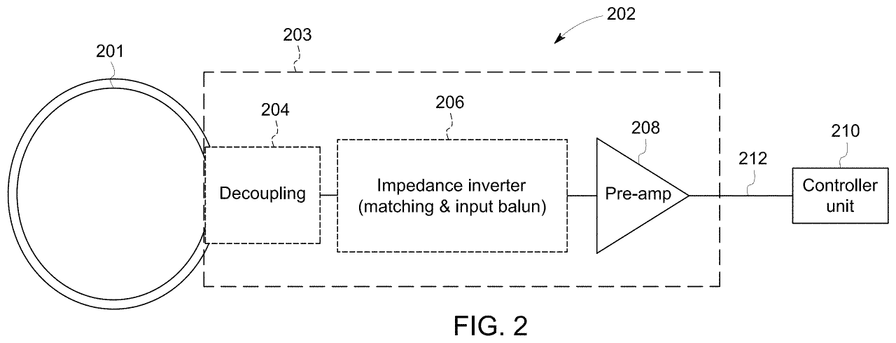

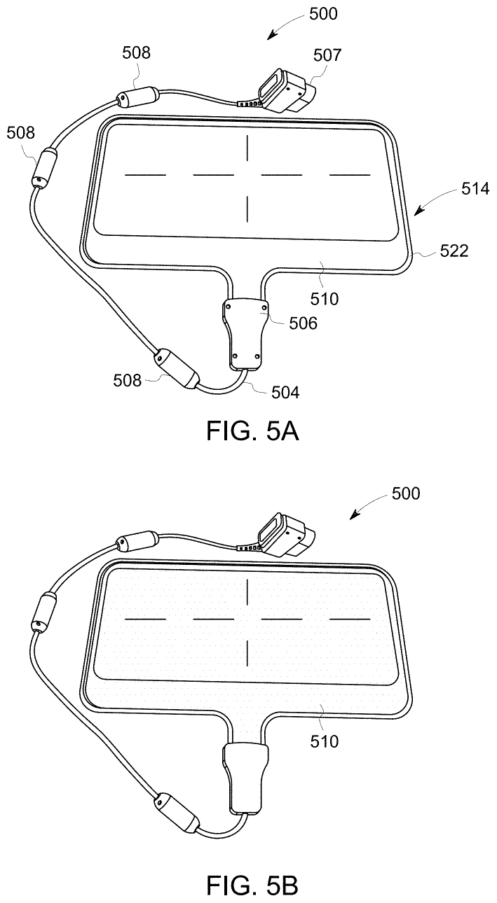

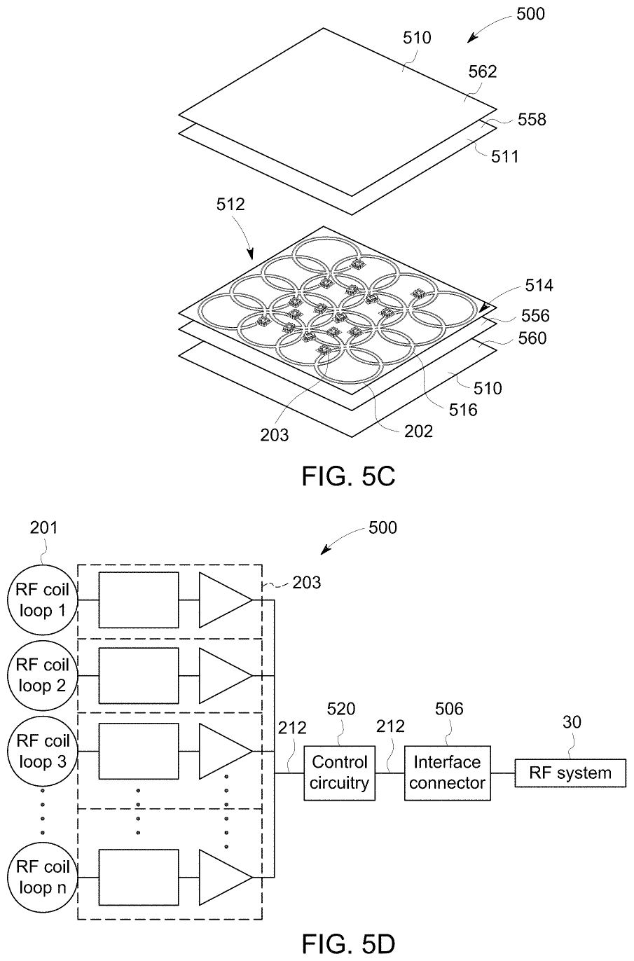

Turning now to FIG. 2, a schematic view of an RF coil 202 that includes a coil loop 201 coupled to a controller unit 210 via a coupling electronics portion 203 and a coil-interfacing cable 212 is shown. In one example, the RF coil may be a surface receive coil, which may be single- or multi-channeled. RF coil 202 may operate at one or more frequencies in MR system 10. Coil-interfacing cable 212 may be a coil-interfacing cable extending between coupling electronics portion 203 and an interfacing connector of an RF coil array or an RF coil array interfacing cable extending between the interfacing connector of the RF coil array and other components of MR system 10 such as RF system 30 (see FIG. 5D described later).

Two coil loops can couple magnetically and electrically. One form of coupling is mutual inductance where signal and noise are transferred from one coil loop to another. The mutual inductance may be reduced by overlapping the coil loops. The mutual inductance may also be reduced by using a high blocking impedance in the coupling electronics portion. The blocking impedance R_{block} seen by the coil loop in general depends on the resistance R of the coil loop, matching characteristic impedance Z₀ of the transmission line, and input impedance of the LNA (a linear amplifier or preamplifier) Rₗₙₐ and may be approximated as:

$${R}_{block} = \frac{{Z}_{0} R}{{R}_{lna}} .$$

When a relatively-high blocking impedance R_{block} is used,

$$\frac{{X}_{L}}{R} \leq \frac{{Z}_{0}}{{R}_{lna}}$$

and the induced current from one coil loop to another is minimized, where X_{L}=ω₀L is the reactance of the coil loop at the resonance frequency of the coil loop.

Other coupling such as coupling through electric field and eddy current may be minimized by reducing the cross-section of the wire conductor in coil loop 201.

The coupling electronics portion 203 may be coupled to coil loop 201 of RF coil 202. Herein, coupling electronics portion 203 may include a decoupling circuit 204, impedance inverter circuit 206, and a pre-amplifier 208. Decoupling circuit 204 may effectively decouple the RF coil during a transmit operation. Typically, RF coil 202 in the receive mode may be positioned adjacent a body of a subject being imaged by MR system 10 in order to receive echoes of the RF signal transmitted during the transmit mode. If RF coil 202 is not used for transmission, RF coil 202 is decoupled from the RF transmit coil such as the RF body coil while the RF transmit coil is transmitting the RF signal. The decoupling of the receive coil from the transmit coil may be achieved using resonance circuits and PIN diodes, microelectromechanical systems (MEMS) switches, or another type of switching circuitry. Herein, the switching circuitry may activate detuning circuits operatively connected to RF coil 202.

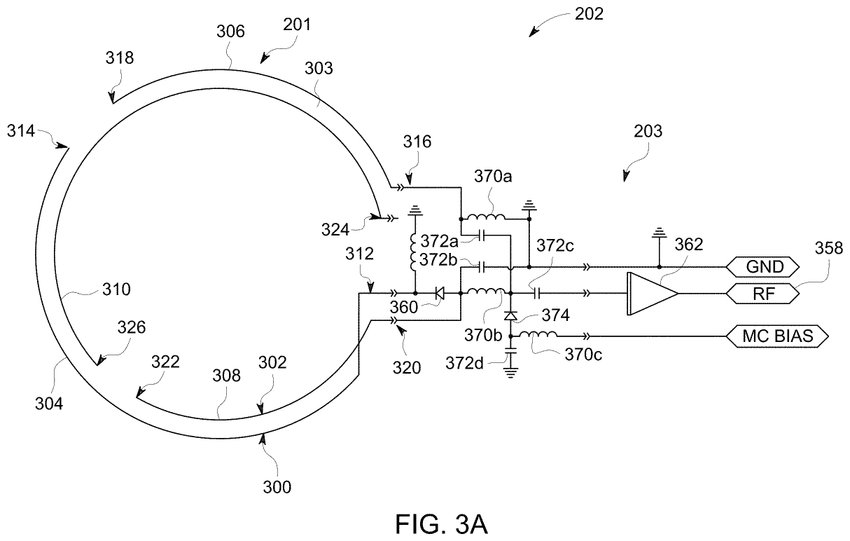

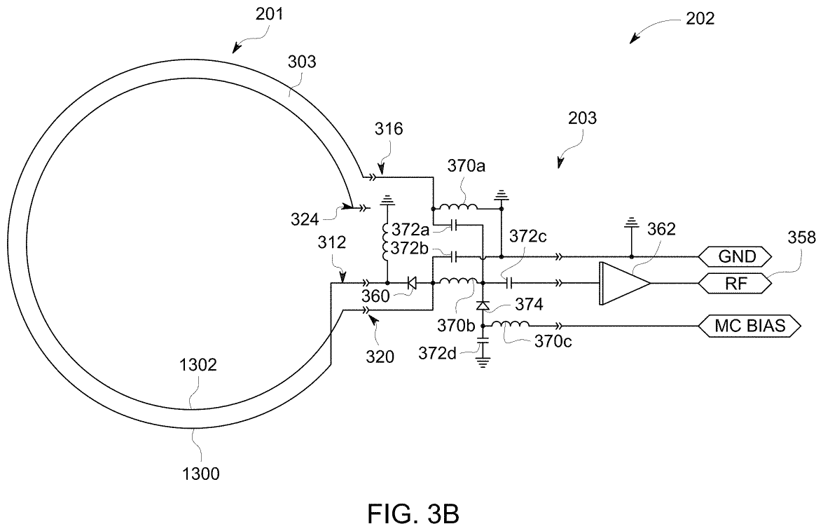

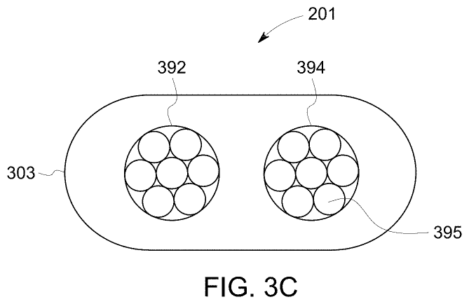

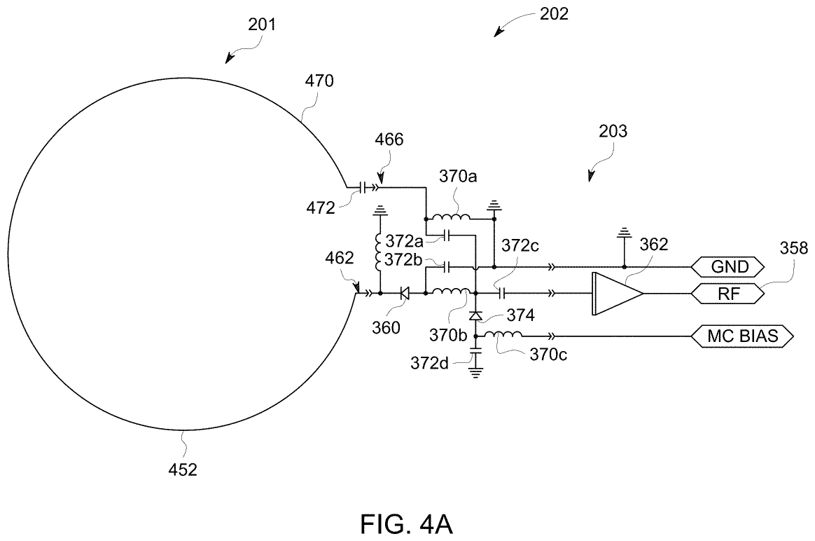

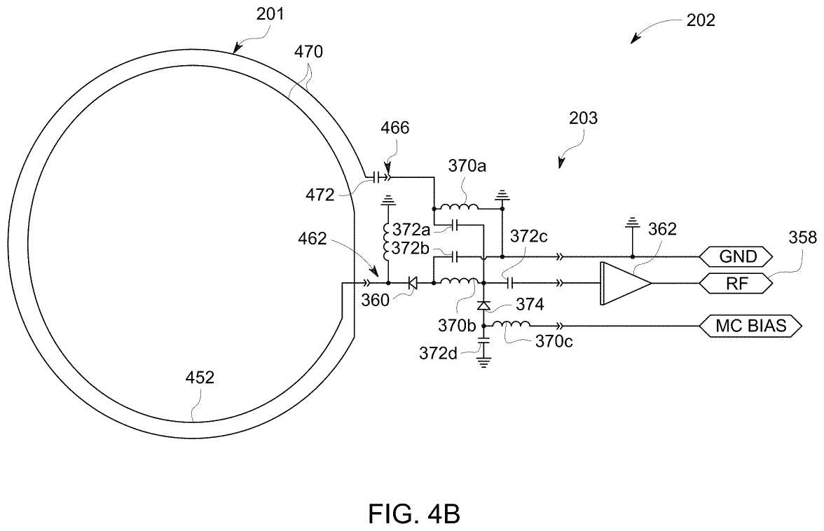

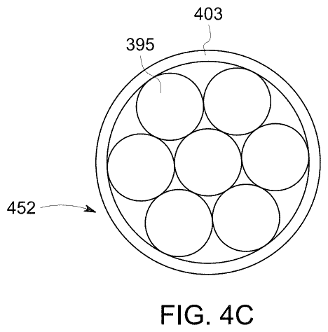

The impedance inverter circuit 206 may form an impedance matching network between RF coil 202 and pre-amplifier 208. Impedance inverter circuit 206 is configured to transform a coil impedance of RF coil 202 into an optimal source of impedance for pre-amplifier 208. The impedance inverter circuit 206 may include an impedance matching network and an input balun. Pre-amplifier 208 receives MR signals from corresponding RF coil 202 and amplifies the received MR signals. In one example, the pre-amplifier may have a low input impedance that is configured to accommodate a relatively high blocking or source impedance. Additional details regarding the RF coil and associated coupling electronics portion will be explained in more detail below with respect to FIGS. 3A, 3B, 4A, and 4B. coupling electronics portion 203 may be packaged in a small PCB with a surface area of approximately 2 cm² or smaller. The PCB may be protected with a conformal coating or an encapsulating resin.

The coil-interfacing cable 212, such as a RF coil array interfacing cable, may be used to transmit signals between the RF coils and other components of MR system 10. The RF coil array interfacing cables may be disposed within the bore or imaging space of MR system 10 and subjected to electro-magnetic fields produced and used by MR system 10. In MR systems, coil-interfacing cables, such as coil-interfacing cable 212, may support transmitter-driven common-mode currents, which may in turn create field distortions and/or unpredictable heating of components. Typically, common-mode currents are blocked by using baluns. Baluns or common-mode traps provide high common-mode impedances, which in turn reduces the effect of transmitter-driven currents.

Thus, coil-interfacing cable 212 may include one or more baluns. In traditional coil-interfacing cables, baluns are positioned with a relatively high density, as high dissipation/voltages may develop if the balun density is too low or if baluns are positioned at an inappropriate location. However, this dense placement may adversely affect flexibility, cost, and performance. As such, the one or more baluns in the coil-interfacing cable may be continuous baluns to ensure no high currents or standing waves, independent of positioning. The continuous baluns may be distributed, flutter, and/or butterfly baluns.

FIG. 3A is a schematic of an exemplary RF coil 202 having segmented conductors formed in accordance with an embodiment. RF coil 202 is a non-limiting example of RF coil 202 shown in FIG. 2 and as such includes coil loop 201 and coupling electronics portion 203. The coupling electronics portion allows the RF coil to transmit and/or receive RF signals when driven by RF system 30 (shown in FIG. 1). In the illustrated embodiment, RF coil 202 includes a first conductor 300 and a second conductor 302. first and second conductors 300, 302 may be segmented such that the conductors form an open circuit (e.g., form a monopole). The segments of conductors 300, 302 may have different lengths. The length of first and second conductors 300, 302 may be varied to achieve a select distributed capacitance, and accordingly, a select resonance frequency.

The first conductor 300 includes a first segment 304 and a second segment 306. First segment 304 includes a driven end 312 at an interface terminating to coupling electronics portion 203, which will be described in more detail below. First segment 304 also includes a floating end 314 that is detached from a reference ground, thereby maintaining a floating state. Second segment 306 includes a driven end 316 at the interface terminating to the coupling electronics portion and a floating end 318 that is detached from a reference ground.

The second conductor 302 includes a first segment 308 and a second segment 310. First segment 308 includes a driven end 320 at the interface. First segment 308 also includes a floating end 322 that is detached from a reference ground, thereby maintaining a floating state. Second segment 310 includes a driven end 324 at the interface, and a floating end 326 that is detached from a reference ground. Driven end 324 may terminate at the interface such that end 324 is only coupled to the first conductor through the distributed capacitance. The capacitors shown around the loop between the conductors represent the capacitance between the wire conductors.

Distributed capacitance (DCAP), as used herein, represents a capacitance exhibited between conductors that grows evenly and uniformly along the length of the conductors and is void of discrete or lumped capacitive components and discrete or lumped inductive components. In the examples herein, the capacitance may grow in a uniform manner along the length of first and second conductors 300, 302. For example, first conductor 300 exhibits a distributed capacitance that grows based on the length of first and second segments 304, 306. Second conductor 302 exhibits a distributed capacitance that grows based on the length of first and second segments 308, 310. First segments 304, 308 may have a different length than second segments 306, 310. The relative difference in length between first segments 304, 308 and second segments 306, 310 may be used to produce an effective LC circuit have a resonance frequency at the desired center frequency. For example, by varying the length of first segments 304, 308 relative to the lengths of second segments 306, 310, an integrated distributed capacitance may be varied.

In the illustrated embodiment, the first and second wire conductors 300, 302 are shaped into a coil loop that terminates to an interface. But in other embodiments, other shapes are possible. For example, the coil loop may be a polygon, shaped to conform the contours of a surface (e.g., housing), and/or the like. The coil loop defines a conductive pathway along the first and second conductors. The first and second conductors are void of any discrete or lumped capacitive or inductive elements along an entire length of the conductive pathway. The coil loop may also include loops of varying gauge of stranded or solid conductor wire, loops of varying diameters with varying lengths of the first and second conductors 300, 302, and/or loops of varying spacing between the first and second conductors. For example, each of the first and second conductors may have no cuts or gaps (no segmented conductors) or one or more cuts or gaps (segmented conductors) at various locations along the conductive pathway.

A dielectric material 303 encapsulates and separates first and second conductors 300, 302. Dielectric material 303 may be selectively chosen to achieve a select distributive capacitance. Dielectric material 303 may be based on a desired permittivity E to vary the effective capacitance of the coil loop. For example, the dielectric material 303 may be air, rubber, plastic, or any other dielectric material. In one example, the dielectric material may be polytetrafluoroethylene (pTFE). For example, dielectric material 303 may be an insulating material surrounding the parallel conductive elements of first and second conductors 300, 302. Alternatively, first and second conductors 300, 302 may be twisted upon one another to form a twisted pair cable. As another example, dielectric material 303 may be a plastic material. First and second conductors 300, 302 may form a coaxial structure in which plastic dielectric material 303 separates the first and second conductors. As another example, the first and second conductors may be configured as planar strips.

The coupling electronics portion 203 is operably and communicatively coupled to RF system 30 to allow RF coil 202 to transmit and/or receive RF signals. In the illustrated embodiment, coupling electronics portion 203 includes a signal interface 358 configured to transmit and receive the RF signals. Signal interface 358 may transmit and receive the RF signals via a cable. The cable may be a 3-conductor triaxial cable having a center conductor, an inner shield, and an outer shield. The center conductor is connected to the RF signal and pre-amp control (RF), the inner shield is connected to ground (GND), and the outer shield is connected to the multi-control bias (diode decoupling control) (MC_BIAS). A 10V power connection may be carried on the same conductor as the RF signal.

As explained above with respect to FIG. 2, coupling electronics portion 203 includes a decoupling circuit, impedance inverter circuit, and pre-amplifier. As illustrated in FIG. 3A, the decoupling circuit includes a decoupling diode 360. Decoupling diode 360 may be provided with voltage from MC_BIAS, for example, in order to turn decoupling diode 360 on. When on, decoupling diode 360 causes conductor 300 to short with conductor 302, thus causing the coil be off-resonance and hence decouple the coil during a transmit operation, for example.

The impedance inverter circuit includes a plurality of inductors, including first inductor 370a, second inductor 370b, and third inductor 370c; a plurality of capacitors, including first capacitor 372a, a second capacitor 372b, a third capacitor 372c, and a fourth capacitor 372d; and a diode 374. The impedance inverter circuit includes matching circuitry and an input balun. As shown, the input balun is a lattice balun that includes first inductor 370a, second inductor 370b, first capacitor 372a, and second capacitor 372b. In one example, diode 374 limits the direction of current flow to block RF receive signals from proceeding into decoupling bias branch (MC_BIAS).

The pre-amplifier 362 may be a low input impedance pre-amplifier that is optimized for high source impedance by the impedance matching circuitry. The pre-amplifier may have a low noise reflection coefficient, γ, and a low noise resistance, Rn. In one example, the pre-amplifier may have a source reflection coefficient of γ substantially equal to 0.0 and a normalized noise resistance of Rn substantially equal to 0.0 in addition to the low noise figure. However, γ values substantially equal to or less than 0.1 and Rn values substantially equal to or less than 0.2 are also contemplated. With the pre-amplifier having the appropriate γ and Rn values, the pre-amplifier provides a blocking impedance for RF coil 202 while also providing a large noise circle in the context of a Smith Chart. As such, current in RF coil 202 is minimized, the pre-amplifier is effectively noise matched with RF coil 202 output impedance. Having a large noise circle, the pre-amplifier yields an effective signal to noise ratio (SNR) over a variety of RF coil impedances while producing a high blocking impedance to RF coil 202.

In some examples, pre-amplifier 362 may include an impedance transformer that includes a capacitor and an inductor. The impedance transformer may be configured to alter the impedance of the pre-amplifier to effectively cancel out a reactance of the pre-amplifier, such as capacitance caused by a parasitic capacitance effect. Parasitic capacitance effects can be caused by, for example, a PCB layout of the pre-amplifier or by a gate of the pre-amplifier. Further, such reactance can often increase as the frequency increases. Advantageously, however, configuring the impedance transformer of the pre-amplifier to cancel, or at least minimize, reactance maintains a high impedance (i.e. a blocking impedance) to RF coil 202 and an effective SNR without having a substantial impact on the noise figure of the pre-amplifier. The lattice balun described above may be a non-limiting example of an impedance transformer.

In examples, the pre-amplifier described herein may a low input pre-amplifier. For example, in some embodiments, a “relatively low” input impedance of the preamplifier is less than approximately 5 ohms at resonance frequency. The coil impedance of RF coil 202 may have any value, which may be dependent on coil loading, coil size, field strength, and/or the like. Examples of the coil impedance of RF coil 202 include, but are not limited to, between approximately 2 ohms and approximately 10 ohms at 1.5 T magnetic field strength, and/or the like. The impedance inverter circuitry is configured to transform the coil impedance of RF coil 202 into a relatively high source impedance. For example, in some embodiments, a “relatively high” source impedance is at least approximately 100 ohms and may be greater than 150 ohms.

The impedance transformer may also provide a blocking impedance to RF coil 202. Transformation of the coil impedance of RF coil 202 to a relative high source impedance may enable the impedance transformer to provide a higher blocking impedance to RF coil 202. Exemplary values for such higher blocking impedances include, for example, a blocking impedance of at least 500 ohms, and at least 1000 ohms.

FIG. 3B is a schematic of another exemplary RF coil 202 and coupling electronics portion 203 according to another embodiment. The RF coil of FIG. 3B is a non-limiting example of RF coil 202 and coupling electronics shown FIG. 2, and as such includes a coil loop 201 and coupling electronics portion 203. RF coil 202 includes a first conductor 1300 in parallel with a second conductor 1302. Different from RF coil 202 shown in FIG. 3A that includes segmented conductors 300, 302, at least one of first and second conductors 1300, 1302 are elongated and continuous.

In the illustrated embodiment, first and second conductors 1300, 1302 are uninterrupted and continuous along an entire length of the coil loop. The coil loop may also include loops of varying gauge of stranded or solid conductor wire, loops of varying diameters with varying lengths of first and second conductors 1300, 1302, and/or loops of varying spacing between the first and second conductors.

The first and second conductors 1300, 1302 have a distributed capacitance along the length of the coil loop (e.g., along the length of first and second conductors 1300, 1302). First and second conductors 1300, 1302 exhibit a substantially equal and uniform capacitance along the entire length of the coil loop. In the examples herein, the capacitance may grow in a uniform manner along the length of first and second conductors 1300, 1302. At least one of first and second conductors 1300, 1302 are elongated and continuous. In the illustrated embodiment, both first and second conductors 1300, 1302 are elongated and continuous. But in other embodiments, only one of first or second conductors 1300, 1302 may be elongated and continuous. First and second conductors 1300, 1302 form continuous distributed capacitors. The capacitance grows at a substantially constant rate along the length of conductors 1300, 1302. In the illustrated embodiment, first and second conductors 1300, 1302 form elongated continuous conductors that exhibits DCAP along the length of first and second conductors 1300, 1302. First and second conductors 1300, 1302 are void of any discrete capacitive and inductive components along the entire length of the continuous conductors between terminating ends of first and second conductors 1300, 1302. For example, first and second conductors 1300, 1302 do not include any discrete capacitors, or any inductors along the length of the coil loop.

FIG. 3C shows a cross-sectional view of an exemplary coil loop 201. Coil loop 201 includes first wire conductor 392 and second wire conductor 394 surrounded by and encapsulated in dielectric material 303. Wire conductors 392, 394 may be conductors 300, 302, 1300, 1302 described above. Each wire conductor may have a suitable cross-sectional shape, herein a circular cross-sectional shape. However, other cross-sectional shapes for the wire conductors are possible, such as elliptical, cylindrical, rectangular, triangular, or hexagonal. The wire conductors may be separated by a suitable distance, and the distance separating the conductors as well as the diameters of the wire conductors may be selected to achieve a desired capacitance. Further, each of first wire conductor 392 and second wire conductor 394 may be a multi-strand wire conductor, which has a plurality of strands 395, such as a seven conductor stranded wire (e.g., having seven stranded wires), but solid conductors may also be used instead of stranded wire. Stranded wire may provide more flexibility relative to solid conductors, at least in some examples.

As appreciated by FIGS. 3A and 3B, the two parallel conductors including the coil loop of an RF coil may each be continuous conductors, as illustrated in FIG. 3B, or one or both of the conductors may be non-continuous, as illustrated in FIG. 3A. For example, both conductors shown in FIG. 3A may include cuts, resulting in each conductor having two segments. The resulting space between conductor segments may be filled with the dielectric material that encapsulates and surrounds the conductors. The two cuts may be positioned at different locations, e.g., one cut at 135° and the other cut at 225° (relative to where the coil loop interfaces with the coupling electronics). By including discontinuous conductors, the resonance frequency of the coil may be adjusted relative to a coil that includes continuous conductors. In an example, an RF coil that includes two continuous parallel conductors encapsulated and separated by a dielectric, the resonance frequency may be a smaller, first resonance frequency. If that RF coil instead includes one discontinuous conductor (e.g., where one of the conductors is cut and filled with the dielectric material) and one continuous conductor, with all other parameters (e.g., conductor wire gauge, loop diameter, spacing between conductors, dielectric material) being the same, the resonance frequency of the RF coil may be a larger, second resonance frequency. In this way, parameters of the coil loop, including conductor wire gauge, loop diameter, spacing between conductors, dielectric material selection and/or thickness, and conductor segment number and lengths, may be adjusted to tune the RF coil to a desired resonance frequency.

FIGS. 4A and 4B shows more exemplary RF coils 202. FIG. 4C is a cross-sectional view of a wire conductor 452 used in coil loop 201 of RF coils 202. Different from coil loops 201 shown in FIGS. 3A-3C that include first conductor 300, 1300 and second conductor 302, 1302 and two driven ends at each end of the conductors, coil loops 201 shown in FIGS. 4A-4B includes one single wire conductor 452 and one driven end 462, 466 at each end of wire conductor 452. Coil loop 201 may form into one turn 470 (FIG. 4A) or a plurality of turns 470 (FIG. 4B). The resistance of coil loop 201 increases approximately by number of turns 470, and the loop loss increases approximately by square root of the number of turns 470, while the body loss increases approximately by the number of turns 470. As a result, the SNR of coil loop 201 is increased approximately by the square root of the number of turns. In other words, multiple turns are used to increase the ratio of the body loss over the loop loss, compared with a single turn coil loop. Coil loop 201 forms into a shape of a circle, and may form into other shapes such as a polygon, oval, or irregular shapes. Coil loop 201 defines a conductive pathway along wire conductor 452. Wire conductor 452 is shown as uninterrupted and continuous along an entire length of the coil loop. The coil loop may also include loops of varying gauge of stranded or solid conductor wire, loops of varying diameters with varying lengths of conductors 452. For example, conductor 452 may have no cuts or gaps (no segmented conductors) or one or more cuts or gaps (segmented conductors) at various locations along the conductive pathway. One or more capacitors 472 may be placed at the cuts, gaps, or at the end of the coil loop. The capacitance of capacitors 472 may be variable.

FIG. 4C shows a cross-sectional view of wire conductor 452. In the exemplary embodiment, conductor 452 has a suitable cross-sectional shape, such as circular, elliptical, rectangular, triangular, or other shapes that enable conductor 452 functions as described herein. Insulating material 403 surrounds conductors 452. Dielectric material 403 may be rubber, plastic, or any other dielectric material. conductor 452 includes one or a plurality of strands 395. For example, conductor 452 is a single-strand wire conductor. Alternatively, conductor 452 is a multi-strand wire conductor having a plurality of strands 395, where an individual strand 395 may be surrounded by insulating material or not surrounded by insulating material. Individual strands 395 may be twisted upon each other or may be parallel to each other, along the length of strand 395. In one example, wire conductor 452 includes 19 strands that are 36 AWG each for an overall thickness of 24 AWG, and the cross section of wire conductor 452 has a diameter of 0.025 inches (0.06 cm). A coil loop 201 including multi-strand conductors 452 has a higher penetration depth and a higher SNR than a coil loop 201 of the same diameter that includes distributed capacitance wire conductors 300, 302, 1300, 1302. Therefore, the size of coil loop 201 may be reduced by including multi-strand wire conductors 452 instead of distributed capacitance wire conductors 300, 302, 1300, 1302 for the same penetration depth, and consequently an increase number of RF coils 202 may be included in a coil array.

Referring back to FIGS. 2, 3A, 3B, 4A, and 4B, coil loop 201 is coupled to coupling electronics portion 203. Coupling electronics portion 203 may be the same coupling electronics described above with respect to FIGS. 2, 3A, 3B, 4A, and 4B, and hence like reference numbers are given to like components and further description is dispensed with.

The RF coils 202 presented above with respect to FIGS. 2, 3A, 3B, 4A, and 4B may be used in order to receive MR signals during an MR imaging session. As such, the RF coils of FIGS. 2, 3A, 3B, 4A, and 4B are configured to be coupled to downstream components of MR system 10. RF coils 202 of FIGS. 2, 3A, 3B, 4A, and 4B may be present in an array of RF coils having various configurations.

FIGS. 5A-5D show an exemplary RF coil assembly 500 that includes RF coils 202 described above. RF coil assembly 500 may be a local RF coil assembly 38 of system 10 (see FIG. 1). FIG. 5A is a top perspective view of RF coil assembly 500. FIG. 5B is a bottom perspective view of RF coil assembly 500. FIG. 5C is an exploded view of RF coil assembly 500. FIG. 5D is a block diagram of RF coil assembly 500.

In the exemplary embodiment, RF coil assembly 500 further includes an RF coil array interfacing cable 504 extending from a coil interfacing connector 506 of the RF coil array 514. RF coil array interfacing cable 504 may be used to connect the RF coil assembly 500 to other components of the MR system 10 such as the RF system 30 through a coil array interfacing connector 507. The RF coil array interfacing cable 504 may include a plurality of baluns 508 or contiguous/continuous distributed baluns (not shown).

In the exemplary embodiment, RF coil assembly 500 further includes an outer enclosure 510, RF coils 202 (FIG. 5C), and a substrate assembly 512. RF coils 202 may form into an RF coil array 514. Each RF coils 202 may include an RF coil loop 201. RF coil loop 201 includes wire conductor 516. Wire conductor 516 may be wire conductors 300, 302, 1300, 1302, 452 described above. Wire conductor 516 forms into coil loop 201. RF coil 202 may also include coupling electronics portion 203. RF coil array 514 is coupled to substrate assembly 512 of flexible fabric material. Sandwiching the RF coil array 514 and substrate assembly 512 is an inner enclosure 511 including a first layer 556 and second layer 558. The material of the inner enclosure may be NOMEX® or other suitable material that provides padding, spacing, and/or flame-retardant properties. An outer enclosure including a first layer 560 and a second layer 562 sandwiches RF coil array 514, substrate assembly 512, and inner enclosure 511. The material of first layer 560 of the outer enclosure 510 may be fabricated from a biocompatible material that is cleanable, thus enabling use of the RF coil array in clinical contexts. Second layer 562 o of the outer enclosure 510 may be fabricated from the deformable material. In this way, the RF coils may be positioned on a top surface of the subject. The RF coils may flex and deform as needed to accommodate patient anatomy. In another embodiment, first layer 556 and second layer 558 of inner enclosure 511 may be eliminated from RF coil assembly 500, such that RF coil array 514 and substrate assembly 512 are sandwiched between a first layer 560 and second layer 562 of padded or deformable material of outer enclosure 510.

In the exemplary embodiment, circular coil loop 201 is depicted as an example only. Coil loop 201 may in other shapes, such as oval, irregularly curved, or rectangular, that enable coil loop 201 to function as described herein. In one example, coil loop 201 is fabricated from a flexible 1.3 millimeter (mm) diameter conductor optimized for zero reactance at 127.73 MHz, the resonance frequency of a 3 T MR system. RF coils 202 may be designed for an MR system 10 having a different field strength, such as 1.5 T. Because wire conductor 300, 302, 1300, 1302, 452 of coil loop 201 is flexible, the shape of coil loop 201 may change and be deformed to conform to a curved anatomy of the subject, such as deforming from being circular to other shapes such as oval, elliptical, or irregular shapes like Pringles® chips. A coil-interfacing cable 212 (FIG. 5D) is connected to and extends from each coupling electronic PCB or coupling electronics portion 203 to coil interfacing connector 506. Coil interfacing connector 506 further couples to other components of MR system 10 such as RF system 30 through RF coil array interfacing cable 504 (see FIGS. 5A and 5B). For example, coil interfacing connector 506 is coupled to coil array interfacing connector 507 and coil array interfacing connector 507 is plugged in a coil interface (not shown) when RF coil assembly 500 is in use, coupling RF coil assembly 500 to the rest of the MR system 10, such as RF system 30.

Coupling electronics portion 203 may include a decoupling circuit, impedance inverter circuit, and a pre-amplifier. The decoupling circuit may effectively decouple an RF coil during a transmit operation. The impedance inverter circuit may form an impedance matching network between an RF coil and the pre-amplifier. The impedance inverter circuit is configured to transform a coil impedance of a RF coil into an optimal source impedance for the pre-amplifier. The impedance inverter circuit may include an impedance matching network and an input balun. The pre-amplifier receives MR signals from a RF coil and amplifies the received MR signals. In one example, the pre-amplifier may have a low input impedance that is configured to accommodate a relatively high blocking or source impedance. The coupling electronics portion 203 may be packaged in a small PCB, for example having an area of approximately 2 cm² or smaller. The PCB may be protected with pad or padding material, a conformal coating, or an encapsulating resin.

Control circuitry 520 (FIG. 5D) is the MC_BIAS for switching RF coils between receive and decoupled modes. Elements of control circuitry 520 are incorporated in both coupling electronics portion 203 and coil interfacing connector 506.

In the exemplary embodiment, RF coil assembly 500 is flexible without increased stress on coil loops 201 and other components of RF coil assembly 500. As used herein, an RF coil assembly is flexible when the RF coil assembly may be flexed or bent to change the shape of the RF coil assembly. The RF coils 202 described above are configured to maintain the performance as RF coil when the RF coils are flexed or bent.

As described above, in MR, signals are acquired by an RF coil. Therefore, an RF coil plays a major role in image quality, such as sign-to-noise ratio (SNR) or image distortion, of images acquired by an MR system. RF coils are desirable to be flexible such that RF coils conform to and are proximate to the anatomy of the subject. On the other hand, the shape and relative positions among components of an RF coil should be maintained to ensure consistency of image quality.

In some known RF coil assemblies, coil loops are coupled to a substrate layer through stiches or other attachment mechanisms, When coil loops are attached to substrate through attachment mechanism, coil loops 201 are under stress at the attachment point, causing coil loop 201 to break and reducing the life of RF coil assemblies. Stiches themselves are also under stress in holding the coil loops together, especially when the coil loops are flexed or bent. As a result, stiches will also break and repair and/or maintenance is needed. Further, attaching through attachment mechanisms like stiches is labor intensive. For example, to stich coil loops to a substrate layer, a special industrial sewing machine is needed to apply stitches around coil loops at circumference or part of circumference of a coil loop. Areas around openings, windows, or gaps are challenging for a sewing machine to maneuver. Operating the sewing machine and setting up the sewing machine demand skills of the operator. Extra care is needed to make sure coil loops or coupling electronics are not damaged during the stitching process.

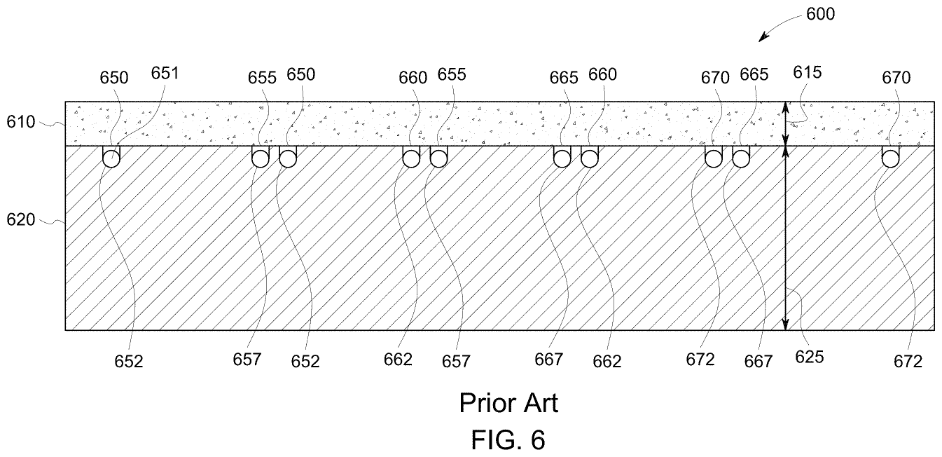

In other known RF coil assemblies, coil loops are positioned in grooves formed in a substrate layer. FIG. 6 shows a cross-sectional view of a known RF coil assembly 600, where coil loops are positioned in grooves formed in a layer 620. RF coil assembly 600 includes a first, outer layer 610. Outer layer 610 is fabricated from one or more sheets of a flexible fabric material. Outer layer 610 may have a first thickness 615. In one example, first thickness 615 may be 1.5 cm or less. RF coil assembly 600 includes a second, inner layer 620. Inner layer 620 is fabricated from a compressible material such as memory foam and may have a second thickness 625. Second thickness 625 may be greater than first thickness 615 and may be 5 cm. Inner layer 620 has a plurality of annular grooves each configured to accommodate an RF coil. Inner layer 620 includes a first annular groove 650. First annular groove 650 accommodates first coil loop 652. For example, first annular groove 650 may be a cut, indentation or groove formed in inner layer 620 that is sized to fit first coil loop 652. When first coil loop 652 is positioned in first annular groove 650, the material including inner layer 620 may surround first coil loop 652, thereby embedding the loop portion of first coil loop 652 in the second inner layer. Inner layer 620 also includes a second annular groove 655 (accommodating second coil loop 657), a third annular groove 660 (accommodating third coil loop 662), a fourth annular groove 665 (accommodating fourth coil loop 667), and a fifth annular groove 670 (accommodating fifth coil loop 672). Coil loops 652, 657, 662, 667, 672 are collectively referred to as coil loops 651. While not shown in FIG. 6, a plurality of rectangular grooves may be present in inner layer 620, each adjacent a respective annular groove. The rectangular grooves may accommodate the coupling electronics portion of each RF coil.

Each annular groove (and hence each RF coil) may be present at a top portion of inner layer 620, and thus a top surface of each RF coil may not be covered by the material of inner layer 620. However, outer layer 610 may cover the top surface of each RF coil. Each of outer layer 610 and inner layer 620 may be compressible, allowing the RF coils embedded therein to conform to a shape of the subject positioned on the RF coil array.

In RF coil assembly 600, layers 610, 620 needs to be compressible such that grooves or indentation may be formed. The thickness of the layers 610, 620 also needs to be at least thicker than coil loop 651 and/or coupling electronics portion. Further, in order to ensure coil loops 651 are embedded in grooves, inner layer 620 is typically not flexible and does allow movement of coil loops 651. In addition, to avoid coil loop 651 and coupling electronics portion from being dislodged from grooves or indentation, layers 610, 620 may need to be attached to one another through attachment mechanisms such as adhesive, which will deteriorate and become ineffective. Because of the thickness of layers 610, 620, RF coil assembly 600 is typically placed under a subject, limiting applications of RF coil assembly 600.

In contrast, in RF coil assembly 500, substrate assemblies are welded together, where substrate assemblies are coupled without a separately-provided fastening mechanism such as attachment mechanisms, e.g. stiches or adhesives, thereby avoiding problems associated with a separately-provided fastening mechanism. In addition, the welded RF coil assemblies and methods described herein provide space for RF coils to reposition and flex without increased stress to the RF coils and fastening mechanism when placing the RF coil assemblies to the subject, thereby increasing image quality of acquired images by increasing conformity of the RF coil with the anatomy of the subject and increasing usable life of the RF coil assembly.

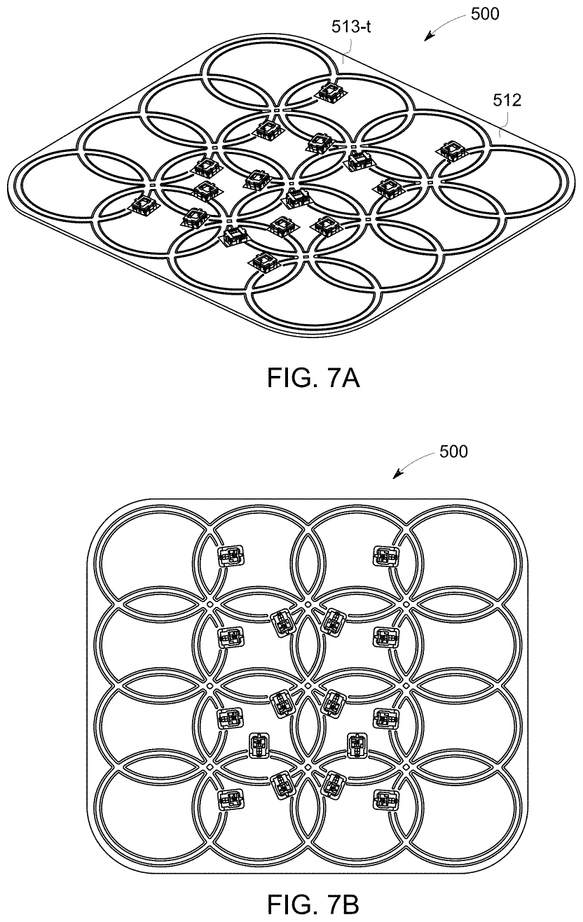

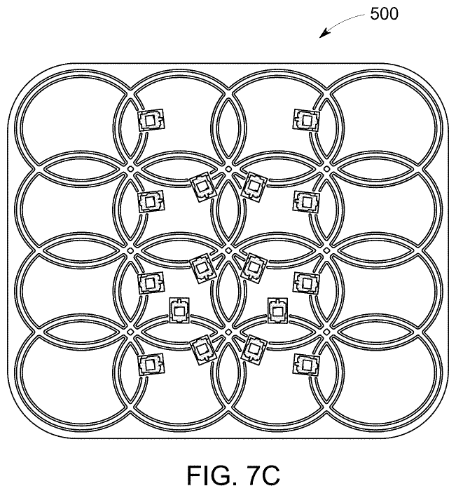

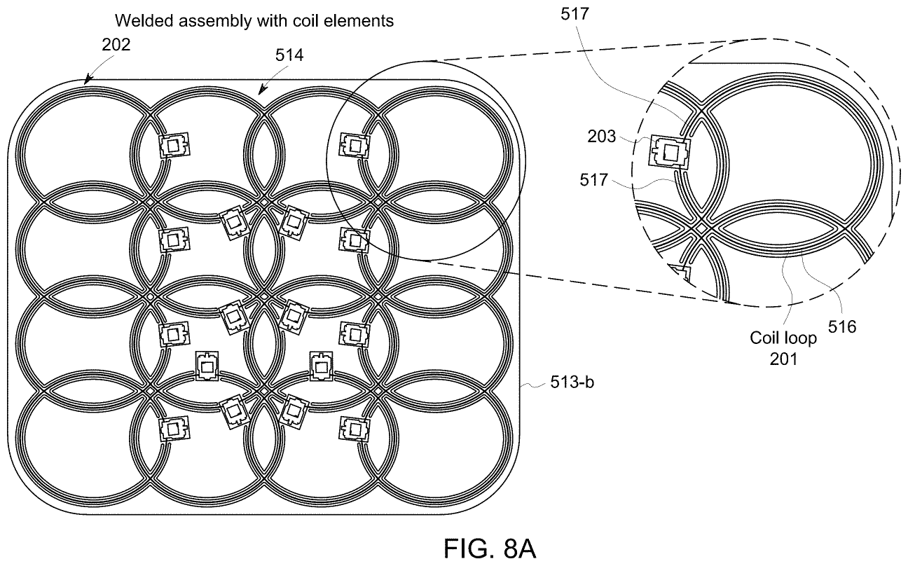

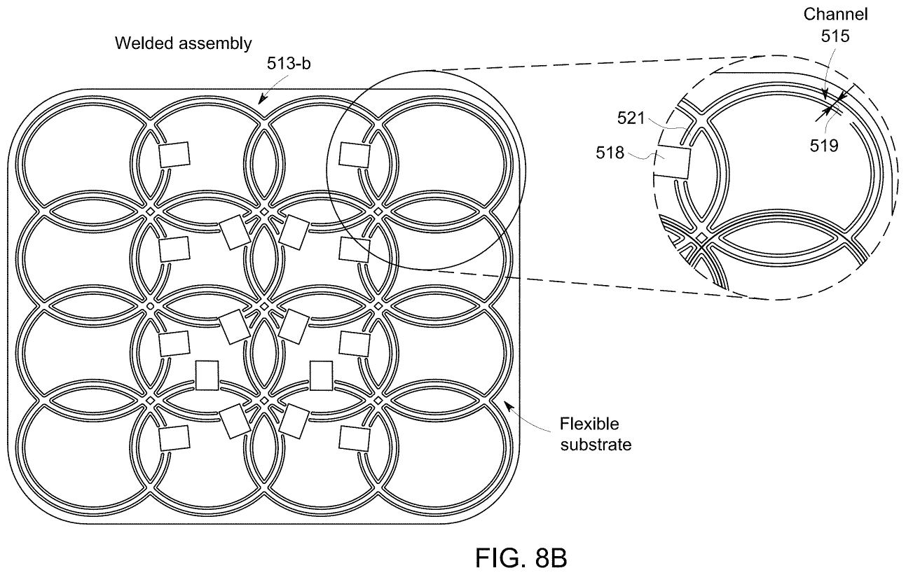

FIGS. 7A-8B show RF coil assembly 500 with outer enclosure 510 and inner enclosure 511 removed and without showing wires and electronics coupled to coupling electronics portions 203. FIG. 7A is a perspective view of RF coil assembly 500. FIG. 7B is a top view of RF coil assembly 500. FIG. 7C is a bottom view of RF coil assembly 500. FIG. 8A shows RF coil assembly 500 shown in FIGS. 7A-7C with a top substrate layer 513-t removed. FIG. 8B shows a bottom substrate layer 513-b without coil loops 201 or coupling electronics portion 203.

In the exemplary embodiment, RF coil assembly 500 includes RF coil array 514 and substrate assembly 512. RF coil array 514 includes one or more RF coils 202. RF coils may be arranged in an array. RF coil 202 includes coil loop 201 formed by wire conductors 516. Wire conductor may be wire conductor 516. RF coil 202 may further include coupling electronics portion 203. Coupling electronics portion 203 is electrically connected to coil loop 201 at ends 517 of wire conductor 516.

In the exemplary embodiment, substrate assembly 512 includes a first substrate layer 513 and a second substrate layer 513. First or second substrate layer 513 may be top or bottom substrate layer 513-t, 513-b. First and second substrate layers 513 are coupled with one another without a separately-provided fastening mechanism, such as attachment mechanism like stitching or adhesive. Substrate assembly 512 is welded, where substrate layers 513 are coupled to one another through welding. For example, substrate assembly 512 is an RF welded substrate assembly, where substrate layers 513 are coupled together through RF welding. Substrate layers 513 are fabricated from RF weldable material such as thermoplastics (e.g. polyvinylchloride and polyurethanes), thermoplastics laminated or coated fabrics, or compound rubber. For example, substrate layers 513 is fabricated from a flexible fabric material, such as DARTEX® material. In RF welding, polar molecules are melted by heat due to movements of polar molecules under RF electric fields, thereby bonding to one another. As such, coupling formed by RF welding is secure. Other welding methods may be used to weld substrate layers 513 to one another. When other welding methods are used, substrate layers 513 are fabricated from material suitable for those welding methods. Similarly, because layers 513 are bonded through fused molecules from heat or solvent during welding, coupling through welding is secure.

In the exemplary embodiment, substrate assembly 512 includes channels 515 (FIG. 8B) when first substrate layer 513 is bonded with second substrate layer 513. Wire conductor 516 is positioned in channel 515. Channel 515 has a width 519 sized to receive wire conductor 516 therein as well as to provide space for movement, repositioning and flexing of wire conductor 516, thereby reducing stress on coil loops 201, increasing usable life of the RF coil assembly 500, and increasing conformity of RF coil assembly with anatomy of the subject. An exemplary width of channel 515 is in the range of 6-8 mm. At ends 521 of channel 515, a gap 518 is formed or defined. Gap 518 may be positioned at other locations of substrate layer 513. Gap 518 is sized to receive coupling electronics portion 203 therein.

In operation, RF coil array 514 is positioned between first and second substrate layers 513. Wire conductors 516 are in channels 515 and may move, reposition, and flex at various dimensions. For example, wire conductors 516 may shift in channel 515. Wire conductors may flex, bend, or rotate with substrate assembly to conform with subject's anatomy. As a result, wire conductors 516 are not under stress from attachment mechanisms, increasing the usable life of RF coil assembly 500. Further, channels 515 and/or gaps 518 may be sealed such that substrate assembly 512 is water-proof to prevent liquid from entering from exterior of substrate assembly 512 into electrical components of RF coil assembly such as coupling electronics portion 203, ensuring performance of RF coil assembly.

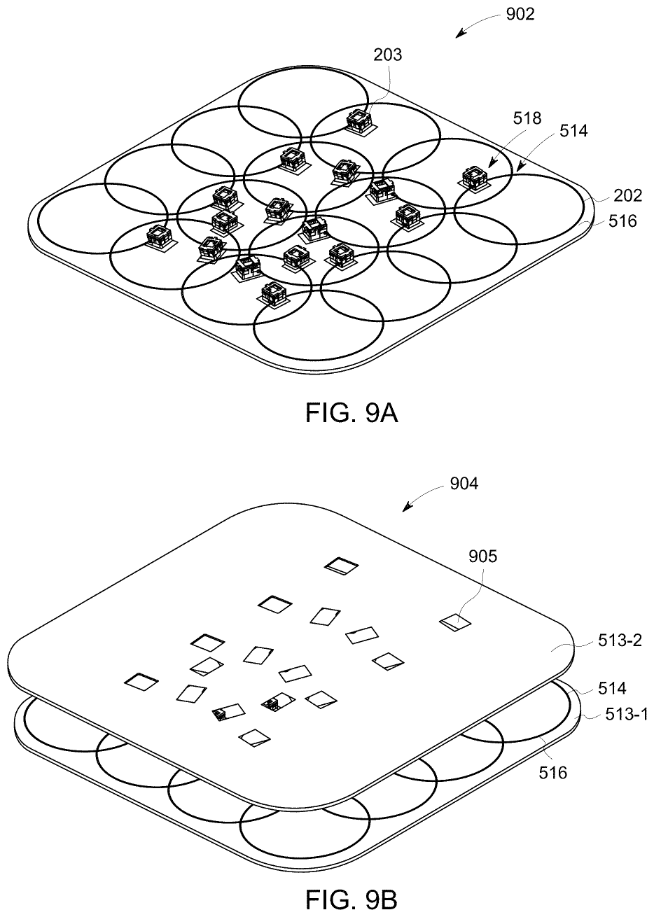

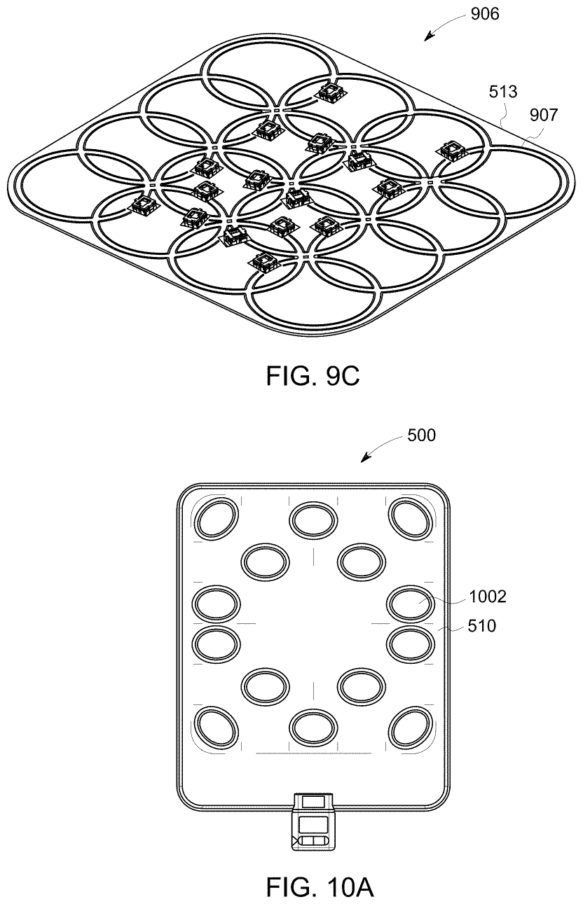

FIGS. 9A-9C illustrate an exemplary method 900 of assembling RF coil assemblies. RF coil assemblies may be RF coil assemblies 500 described above. In the exemplary embodiment, an RF coil array is positioned 902 on a substrate layer (FIG. 9A). The RF coil array 514 may include RF coils 202 that have wire conductors 516. In some embodiments, RF coil 202 also includes coupling electronics portion 203 electrically connected to wire conductor 516. In other embodiments, RF coil 202 does not include a coupling electronics portion. Alternatively, in positioning 902 an RF coil array, coupling electronics portions 203 are not electrically connected with wire conductor 516 and not placed on substrate layer 513, and ends of wire conductor 516 include a gap 518 for connection with coupling electronics portion 203 later in the process. Method 900 further includes positioning 904 (FIG. 9B) another substrate layer 513-2 over RF coil array 514 and first substrate layer 513-1. Substrate layer 513-1, 513-2 may include apertures 905 corresponding to gaps 518 positioned at ends of the wire conductors such that coupling electronics portions 203 and/or coil-interfacing cable (not shown) connected to and extending from coupling electronic portions 203 or wire conductors 516 may pass through apertures 905. The depicted embodiment shows apertures 905 are on substrate layer 513-2. Apertures 905 may be on either one of substrate layers 513 or on both of substrate layers 513.

In the exemplary embodiment, method 900 further includes coupling 906 (FIG. 9C) the first substrate layer with the second substrate layer by welding the first substrate layer with the second substrate layer. Welding may be RF welding. Welding may be other dielectric welding or dielectric sealing, such as microwave welding. Other welding methods may be used such as ultrasonic welding. Welding as used herein refers to coupling materials with the aid of heat or solvent. Comparing to sewing which may take hours or more, RF welding takes seconds, drastically increasing manufacturing speed. After welding, substrate layers 513 are coupled to one another at the welded seams 907. Gaps between segments of seams 907, such as gap 518, may be sealed such that RF coil assembly 500 is water-proof For examples, sealing may be applied at apertures 905 where coupling electronics portion 203 and/or coil-interfacing cable exiting from substrate layers. Welded coupling itself is secure and water proof because molecules are fused together during welding at seams 907. Referring back to FIGS. 5A-5C, other layers of RF coil assembly 500 may also be welded using the same welding method as in welding substrate layers 513. For example, first layer 560 and second layer 562 of outer enclosure 510 may be RF welded together (FIGS. 5A and 5B) at seams 522. Layers 556, 558 of inner enclosure 511 may also be coupled by RF welding (not shown). The same welding method is used to simplify the manufacturing process where the same machinery is used.

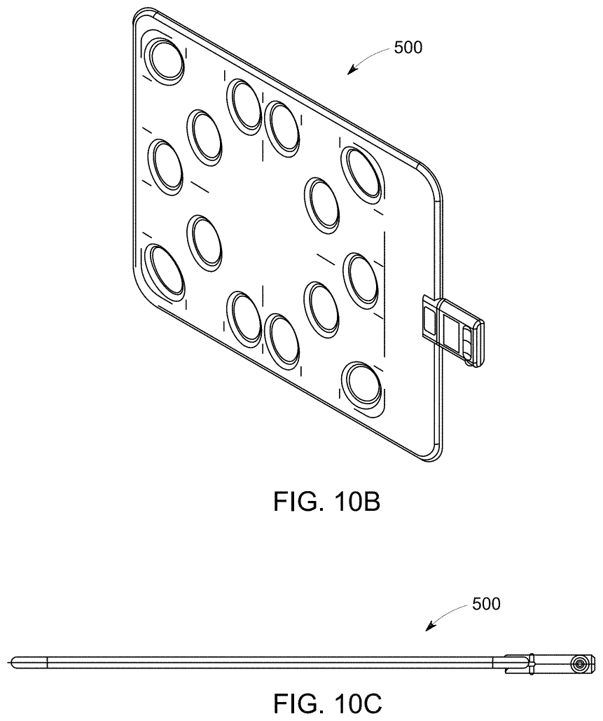

FIGS. 10A-10C shows another exemplary welded RF coil assembly 500. Compared to RF coil assembly 500 shown in FIGS. 5A and 5B, RF coil assembly 500 further includes welded apertures 1002. FIG. 10A is a top or bottom view of RF coil assembly 500. FIG. 10B is a perspective view of RF coil assembly 500. FIG. 10C is a side view of RF coil assembly 500. Outer enclosure 510 may not be breathable and may be uncomfortable for the subject, causing the subject to move, after being placed on the subject for an extended period of time, such as one hour. Motion of a subject deteriorates image quality of acquired MR images. Apertures 1002 allow body heat and perspiration to dissipate from the subject, increasing comfort to the subject. In addition, apertures 1002 provide access to anatomy of the subject for interventional treatments such as biopsy, surgery, or therapy. Apertures 1002 are located within coil loop 201 (see FIG. 8A) such that apertures 1002 do not intersect coil loop 201, coupling electronics portion 203, or a coil-interfacing cable. In other words, the area defined by aperture 1002 is within an area defined or encircled by coil loop 201, or aperture 1002 is enclosed or surrounded by coil loop 201. Apertures 1002 being positioned within a coil loop 201 are advantageous because the anatomy that apertures 1002 provide access to is the same anatomy being imaged by coil loop 201, thereby allowing MR images to be used as guidance for interventional treatments. Apertures 1002 may be constructed before welding, where areas marked for apertures 1002 are removed and edges along apertures 1002 are welded together. Alternatively, apertures 1002 may be constructed after welding, where locations marked for apertures 1002 are welded and then areas surrounded by seams formed by welding are removed.

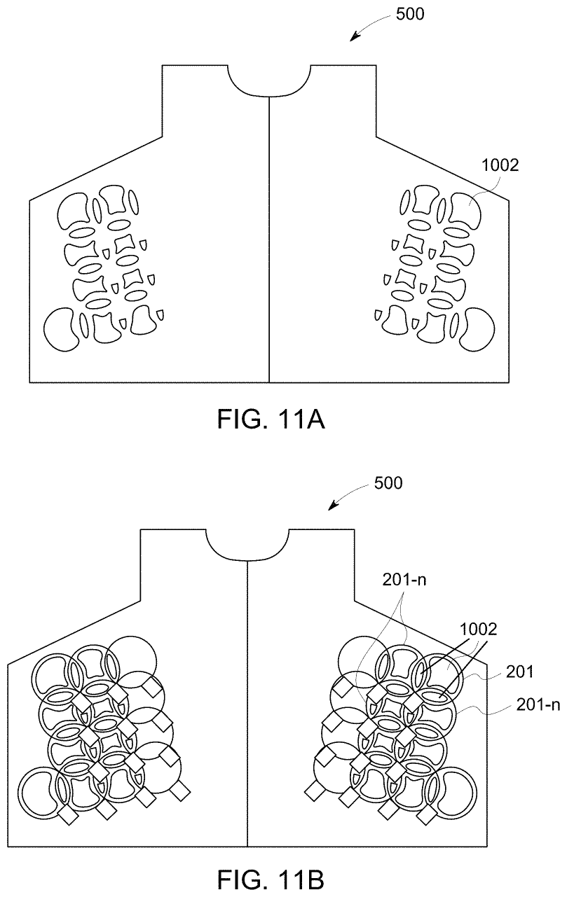

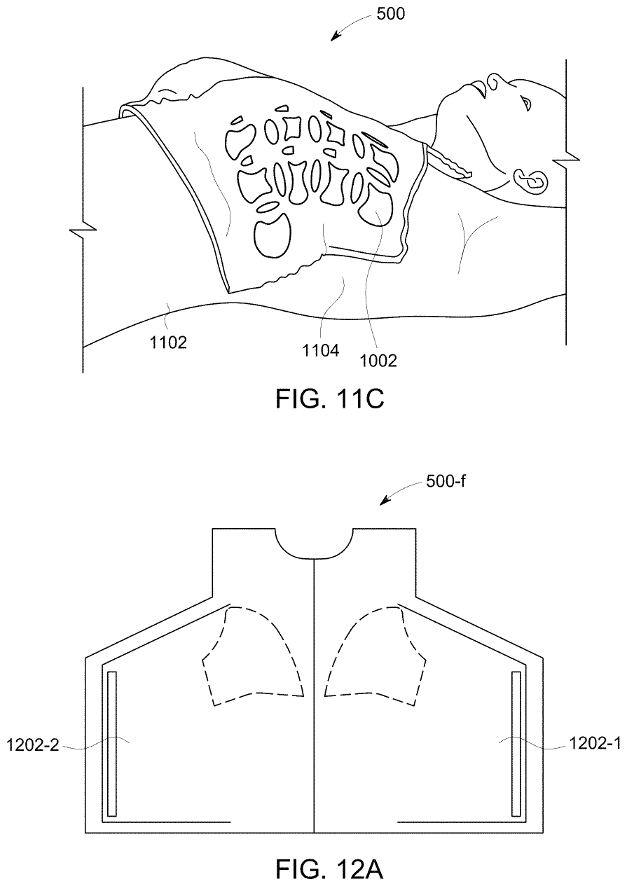

FIGS. 11A-11C show one more exemplary embodiment of RF coil assembly 500. FIG. 11A is a top view of RF coil assembly 500. FIG. 11B is schematic diagram of RF coil assembly 500 with a top substrate layer removed to show coil loops 201. FIG. 11C is a side perspective view of RF coil assembly 500 when being placed on a subject 1102. Similar to RF coil assembly 500 shown in FIGS. 10A-10C, RF coil assembly 500 includes one or more welded apertures 1002. Apertures 1002 are positioned within RF coil loops 201. Within RF coil loop 201, a plurality of apertures 1002 may be included within circumferences defined by RF coil loop 201 and its neighboring RF coil loop 201-n.

In operation, during interventional treatments such as biopsy or surgery, apertures 1002 provide access to anatomy of subject 1102, such as an upper torso 1104 of subject 1102, including breasts, the areas below the armpit, and/or the upper chest area.

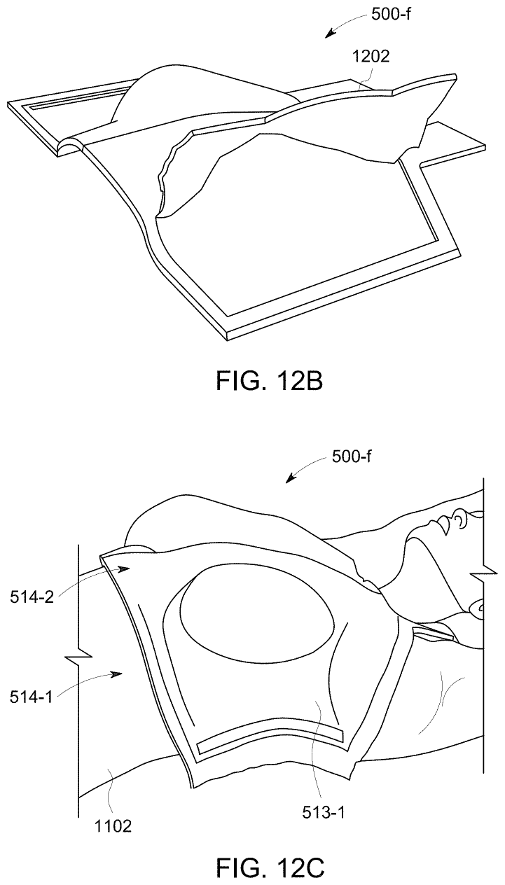

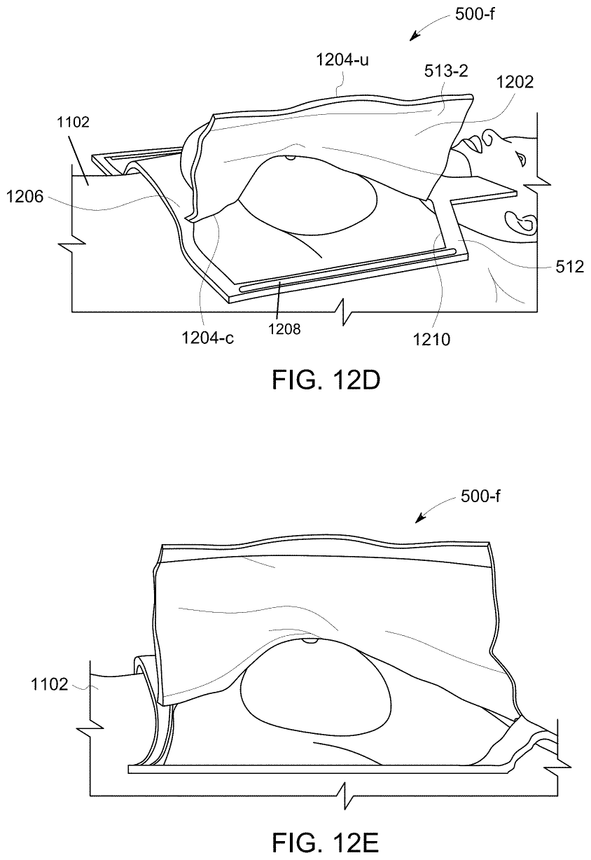

In the depicted embodiment, the coil loop 201 defines a circumference having a diameter of approximately 7 cm. Smaller-sized coil loops are advantageous than larger-sized coil loops because RF coil assembly 500 having smaller-sized coil loops conforms better to contours of the subject and allows to more coil loops than RF coil assembly 500 having larger-sized coil loops, thereby providing faster image acquisition and higher signal-to-noise (SNR) ratio in images. When sizes of the coil loops reduce, space is limited to weld apertures 1002 through RF coil assembly 500. FIGS. 12A-14B show embodiments of RF coil assembly 500-f that includes one or more flaps to provide access to anatomies of subject 1102 during interventional treatments.

FIGS. 12A-12E show an exemplary embodiment of RF coil assembly 500-f. FIG. 12A is top view of RF coil assembly 500-f. FIG. 12B is a perspective view of RF coil assembly 500-f when a flap 1202 is lifted up. FIG. 12C is a perspective view of RF coil assembly 500-f when being placed on subject 1102. FIG. 12D is a perspective view of RF coil assembly 500-f with flap 1202 lifted when being placed on subject 1102. FIG. 12E is side view of RF coil assembly 500-f with flap 1202 lifted when being placed on subject 1102. Different from RF coil assemblies shown in FIGS. 5A-5D and 7A-11C, RF coil assembly 500-f includes flap 1202. Flap 1202 includes a portion of first substrate layer 513-1 (FIG. 12C), a portion of second substrate layer 513-2 (FIG. 12D), and at least one RF coil 202 of RF coil array 514 (see FIG. 5C). RF coils 202 are positioned between first substrate layer 513-1 and second substrate layer 513-2. Flap 1202 includes a coupled side 1204-c and uncoupled sides 1204-u. First substrate layer 513-1 and second substrate layer 513-2 are welded together along uncoupled sides 1204-u, e.g., through RF welding. RF coils 202 of flap 1202 are positioned within coupled side 1204-c and uncoupled sides 1204-u. Flap 1202 is coupled to the remaining 1206 of RF coil assembly 500-f along coupled side 1204-c. A seam 1210 of remaining 1206, where flap 1202 is decoupled from remaining 1206, may be sealed through welding, e.g., RF welding. Flap 1202 is positionable between a closed position (FIGS. 12A and 12C) and an opened position (FIGS. 12B, 12D, and 12E). In the depicted embodiment, RF coil assembly 500-f includes two flaps 1202-1, 1202-2 and two RF coil arrays 514-1, 514-2, RF coil array 514-1 for imaging one part of subject 1102 such as a left breast and its surrounding anatomy and RF coil array 514-2 for imaging another part of subject 1102 such as a right breast and its surrounding anatomy. Flap 1202-1 includes RF coil array 514-1. Flap 1202-2 includes RF coil array 514-2. In some embodiments, RF coil assembly 500-f includes any other number of flaps, such as one, three, or more.

In operation, at a closed position, flap 1202 may be coupled to remaining 1206 through a fastener 1208 such as a hook-and-loop fastener. Fastener 1208 may be welded such as RF welded to substrate assembly 512. Fastener 1208 may be coupled to substrate assembly 512 through other mechanism such as adhesive. At an opened position, flap 1202 is lifted up and may be folded back, providing access to anatomy of subject 1102. Because flap 1202 may be lifted and folded back, RF coil assembly 500-f provides full access to anatomy covered by RF coil array 514-1, 514-2.

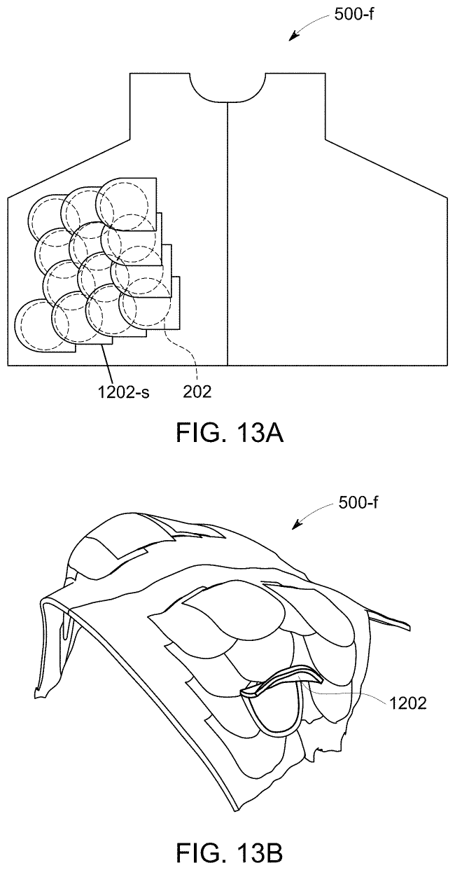

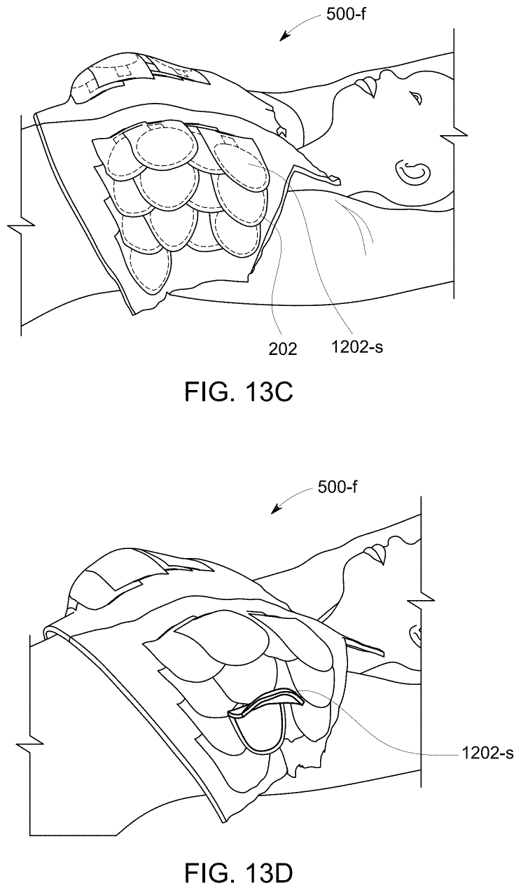

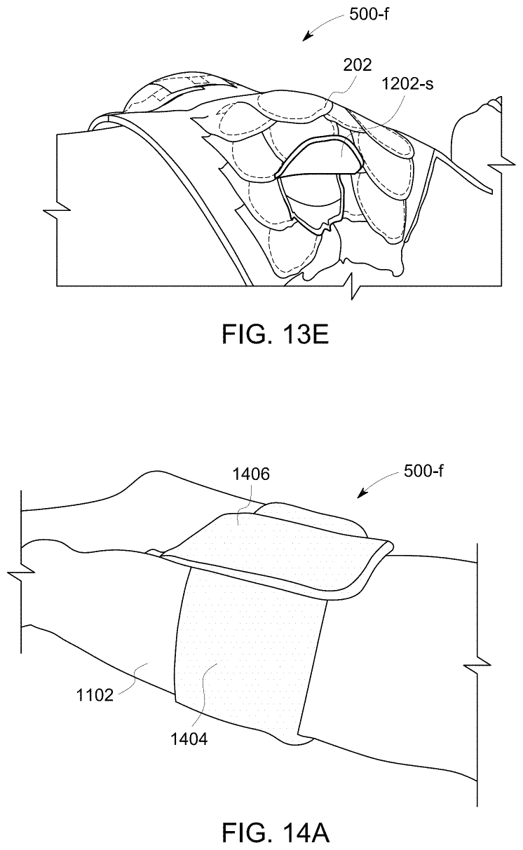

FIGS. 13A-13E show another exemplary embodiment of RF coil assembly 500-f. FIG. 13A is a top view of RF coil assembly 500-f. FIG. 13B is a perspective view of RF coil assembly 500-f with one of flaps 1202 is lifted. FIGS. 13C-13E show RF coil assembly 500-f being placed on subject when flaps are at closed positions (FIG. 13C), and when a flap 1202 is at an opened position (FIGS. 13D and 13E). FIG. 13D is a perspective view. FIG. 13E is a side view. Different from RF coil assembly 500-f shown in FIGS. 12A-12E, where flap 1202 includes an entire RF coil array 514 (see FIG. 5C), flap 1202-s of RF coil assembly 500-f does not include the entire RF coil array 514. In the depicted embodiment, flap 1202-s includes one RF coil 202 of RF coil array 514. In some embodiments, flap 1202-s includes more than one RF coil 202 of RF coil array 514. That is, one RF coil array 514 includes two or more flaps 1202. Flaps 1202 may overlap with one another. In some embodiments, flaps 1202 do not overlap with one another, or some flaps 1202 overlap and some do not. Each flap 1202-s includes a coupled side 1204-c and uncoupled sides 1204-u. Each flap 1202-s is positionable at a closed position (FIG. 13A and FIG. 13C) and at an opened position (FIGS. 13B, 13D, and 13E). Flap 1202-s may be individually lifted and folded back, providing access to anatomy covered by RF coil(s) included in flap 1202-s. Compared to RF coil assembly 500-f shown in FIGS. 12A-12E, opening and closing flaps 1202-s have a reduced effect on the positions of RF coils because a reduced number of RF coils are moved and the moved RF coils are moved at a reduced distance. RF coil assembly 500-f having flap 1202-s also provides increased accuracy in interventional treatments. Individual images of individual RF coil(s) included in flap 1202-s may be used to identify the location of a lesion. If a lesion only appears in an image of one RF coil 202 or images of a few RF coils 202, lesion would be located directly beneath those RF coil(s) 202.

RF coil assemblies 500 shown in FIGS. 11A-13E are configured as a breast coil assembly, which is configured to image at least a portion of an upper torso of subject 1102. Compared to conventional breast coil assembly, which is limited to imaging subject 1102 at a prone position, breast RF coil assembly 500 is configures to image subject 1102 at a supine position, as well as at a prone position. Breast imaging at a supine position is advantageous over imaging at a prone position. Interventional treatments typically are performed when the subject is at a supine position, providing a greater access to the tissue than performing the treatments when the subject is at a prone position, which provides access at the sides of the subject and increase risks of damages to the subject when the lesion such as tumor is positioned away from the sides. Therefore, breast imaging at a supine position provides matching images and anatomy during treatments, allowing guidance to treatments with increased accuracy. Further, conventional breast RF coils are limited to imaging breasts of the subject. In contrast, breast RF coil assembly 500 allows imaging areas other than breasts, such as the areas below the armpit and upper chest area such as level III axillary lymph nodes and supraclavicular lymph nodes. Although supine breast RF coil assembly may be more sensitive to motion such as breathing motion because supine breast RF coil assembly moves with the chest during breathing, the motion effects are largely reduced with increased number of RF coils in the RF coil assembly 500. For example, conventional breast RF coil assembly takes minutes to image the breast. During that period of time, breast moves and patient may also become uncomfortable and shift around. In contrast, supine RF coil assembly may include a large number of RF coils such as 60 RF coils and takes seconds to image the breast areas, drastically reducing motion effects.

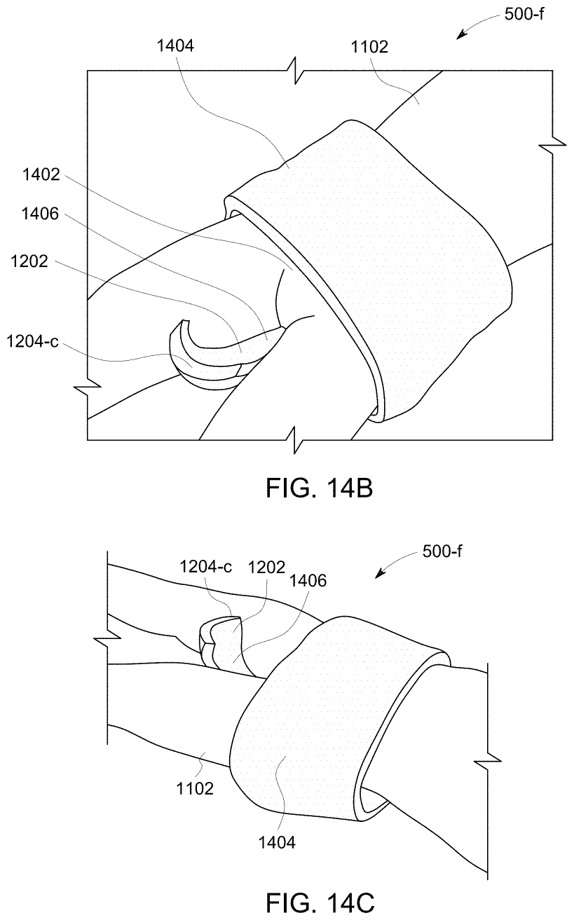

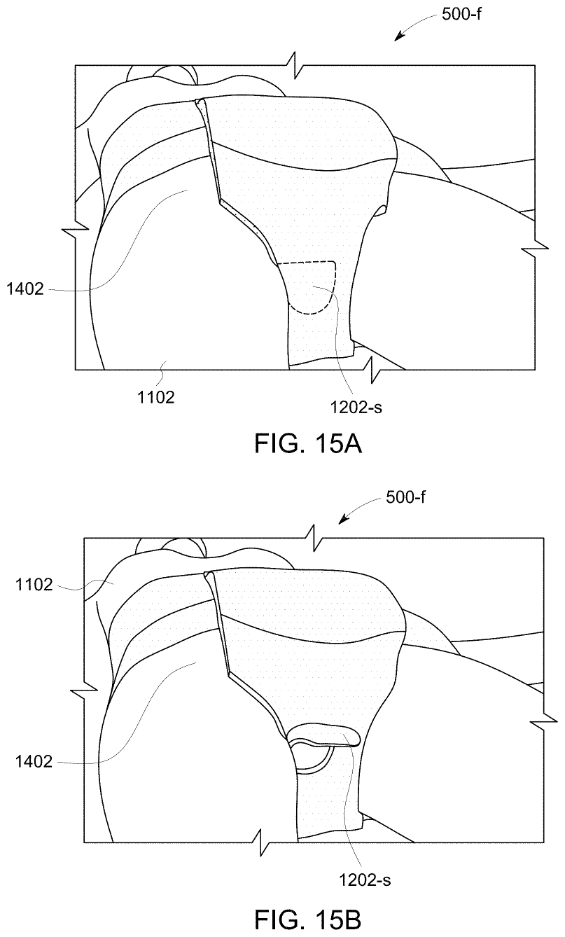

FIGS. 14A-FIG. 15B show exemplary embodiments of RF coil assembly 500-f configured as a pelvic RF coil assembly. A pelvic RF coil assembly is configured to image at least a portion of a pelvis 1402 of subject 1102. Pelvic RF coil assembly 500-f conforms to pelvic contour of subject 1102.

FIGS. 14A-14C show an exemplary embodiment of pelvic RF coil assembly 500-f FIG. 14A is a perspective view of RF coil assembly 500-f when flap 1202 is at a closed position. FIGS. 14B and 14C are perspective views of RF coil assembly 500-f when flap 1202 is at an opened position. FIG. 14B is a perspective view when viewing from a lower torso of subject 1102. FIG. 14B is a perspective view when viewing from an upper torso of subject 1102.

In the exemplary embodiment, RF coil assembly 500-f includes a torso portion 1404 configured to cover a lower torso of subject 1102 and a crotch portion 1406 configured to cover a crotch area of subject 1102. Torso portion 1404 may include an RF coil array 514 (see FIG. 5C). Crotch portion 1406 includes RF coil array 514. Crotch portion 1406 is configured as a flap 1202. Flap 1202 is positionable at a closed position (FIG. 14A or an opened position (FIGS. 14B and 14C). Flap 1202 includes the entire RF coil array of crotch portion 1406. Flap 1202 is coupled to torso portion 1404 at a coupled side 1204-c (not shown). At a closed positioned, flap 1202 is coupled to torso portion 1404 at an uncoupled side 1204-u.

In operation, during imaging, flap 1202 is at a closed portion with flap 1202 coupled to torso portion 1404 at coupled side 1204-c. During interventional treatments, to access anatomy of subject 1102, such as prostate, flap 1202 is decoupled from torso portion 1404 at uncoupled side 1204-u.

FIGS. 15A-15B are perspective views of another embodiment of pelvic RF coil assembly 500-f, viewing from the lower torso of subject 1102. FIG. FIG. 15A shows RF coil assembly 500-f when flap 1202-s is at a closed position. FIG. 15B shows RF coil assembly 500-f when flap 1202-s is at an opened position. Different from RF coil assembly 500-f shown in FIGS. 14A-14C, RF coil assembly 500-f includes flap 1202-s that includes one RF coil or a few RF coils 202 (see FIG. 5C), instead of an entire RF coil array 514 (see FIG. 5C) of crotch portion 1406.

In the depicted embodiment, RF coil assembly 500-f includes one flap 1202-s. RF coil assembly 500-f may include two or more flaps 1202-s, where flaps 1202-s may or may not overlap with one another. Alternatively, some flaps 1202-s overlap and some flaps 1202 do not. Like RF coil assembly 500-f shown in FIGS. 13A-13E, RF coil assembly 500-f reduces position changes of RF coils 202 and increases location accuracy during interventional treatments.

At least one technical effect of the systems and methods described herein includes (a) an RF coil assembly that provides flexibility of coil loops without stressing coil loops; (b) an RF coil assembly that provides secure coupling of coil loops as well as permitting movement of the coil loops; (c) assembling an RF coil assembly through welding; (d) a simplified and quick manufacturing process of an RF coil assembly; (e) an RF coil assembly that provides access to interventional treatments; and (f) an RF coil assembly that includes a flap for access to anatomy when the flap is at an opened position.

Exemplary embodiments of assemblies, systems, and methods of RF coil assemblies are described above in detail. The systems and methods are not limited to the specific embodiments described herein but, rather, components of the systems and/or operations of the methods may be utilized independently and separately from other components and/or operations described herein. Further, the described components and/or operations may also be defined in, or used in combination with, other systems, methods, and/or devices, and are not limited to practice with only the systems described herein.

Although specific features of various embodiments of the invention may be shown in some drawings and not in others, this is for convenience only. In accordance with the principles of the invention, any feature of a drawing may be referenced and/or claimed in combination with any feature of any other drawing.

This written description uses examples to disclose the invention, including the best mode, and to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A radio frequency (RF) coil assembly for a magnetic resonance (MR) system, comprising:
an RF coil array comprising:
one or more RF coils each RF coil comprising:
a coil loop that comprises a wire conductor, the wire conductor formed into the coil loop; and
a substrate assembly comprising:
a first substrate layer; and
a second substrate layer, wherein the first substrate layer is coupled with the second substrate layer at seams without a separately-provided fastening mechanism,
wherein the RF coil array is positioned between the first substrate layer and the second substrate layer.

2. The RF coil assembly of claim 1, wherein the substrate assembly is a welded substrate assembly, and the seams are welded seams.

3. The RF coil assembly of claim 2, wherein the substrate assembly is an RF welded substrate assembly, and the seams are RF welded seams.

4. The RF coil assembly of claim 1, wherein the seams define channels positioned between the first substrate layer and the second substrate layer, and coil loops of the one or more RF coils are positioned in the channels.

5. The RF coil assembly of claim 4, wherein the channels are sized to permit movement of the coil loops.

6. The RF coil assembly of claim 4, wherein the substrate assembly further defines a gap between ends of a channel.

7. The RF coil assembly of claim 6, wherein each RF coil further comprises a coupling electronics portion electrically connected to the coil loop and positioned in the gap.

8. The RF coil assembly of claim 1, further comprising welded apertures through the RF coil assembly, wherein each of the welded apertures is positioned within the coil loop.

9. The RF coil assembly of claim 1, wherein the coil loop is a distributed capacitance coil loop that comprises two parallel wire conductors and a dielectric material encapsulating and separating the two parallel wire conductors.

10. The RF coil assembly of claim 1, wherein the wire conductor comprises a plurality of strands.

11. The RF coil assembly of claim 1, wherein the coil loop comprises a plurality of turns formed by the wire conductor.

12. The RF coil assembly of claim 1, further comprising a flap, wherein the flap comprises:
a portion of the first substrate layer;
a portion of the second substrate layer; and
at least one RF coil of the RF coil array, the at least one RF coil positioned between the portion of the first substrate layer and the portion of the second substrate layer,
wherein the flap is coupled with a remaining of the RF coil assembly and positionable between an opened position and a closed position.

13. The RF coil assembly of claim 12, wherein the flap includes one RF coil of the RF coil array.

14. The RF coil assembly of claim 12, wherein the flap includes a plurality of RF coils of the RF coil array.

15. The RF coil assembly of claim 1, wherein the RF coil assembly is configured as a breast RF coil assembly configured to image at least a portion of an upper torso of a subject.

16. The RF coil assembly of claim 1, wherein the RF coil assembly is configured as a pelvic RF coil assembly configured to image at least a portion of a pelvis of a subject.

17. A method of assembling a radio-frequency (RF) coil assembly of a medical imaging system, comprising:
positioning one or more coil loops on a first substrate layer, wherein each coil loop includes a wire conductor, the wire conductor formed into the coil loop;
positioning a second substrate layer over the one or more coil loops; and
forming a substrate assembly that includes the first substrate layer and the second substrate layer by coupling the first substrate layer with the second substrate layer at seams without a separately-provided fastening mechanism.

18. The method of claim 17, wherein coupling the first substrate layer further comprises coupling the first substrate layer with the second substrate layer by welding the first substrate layer with the second substrate layer to form the seams.

19. The method of claim 18, wherein coupling the first substrate layer further comprises coupling the first substrate layer with the second substrate layer by RF welding the first substrate layer with the second substrate layer.

20. The method of claim 17, wherein the seams define channels positioned between the first substrate layer and the second substrate layer, and coil loops of the one or more RF coils are positioned in the channels.

21. The method of claim 20, wherein the channels are sized to permit movement of the coil loops.

22. The method of claim 20, wherein the substrate assembly further defines a gap between ends of a channel.

23. The method of claim 22, wherein each RF coil further comprises a coupling electronics portion electrically connected to the coil loop and positioned in the gap.

24. The method of claim 17, further comprising forming welded apertures through the RF coil assembly, wherein each of the welded apertures is positioned within the coil loop.

25. A radio frequency (RF) coil assembly for a medical imaging system, comprising:
an RF coil array comprising:
one or more RF coils each RF coil comprising:
a coil loop that comprises a wire conductor, the wire conductor formed into the coil loop; and
a substrate assembly comprising:
a first substrate layer; and
a second substrate layer, wherein the first substrate layer is coupled with the second substrate layer at seams without a separately-provided fastening mechanism,
wherein the RF coil array is positioned between the first substrate layer and the second substrate layer.
